(19) United States
(12) Patent Application Publication
(10) Pub. No.: US 2011/0275661 A1
(43) Pub. Date: Nov. 10, 2011

(54) TRICYCLIC NITROGEN COMPOUNDS USED AS ANTIBACTERIALS

(75) Inventors: Ilaria Giordano, Hertfordshire (GB); Alan Joseph Hennessy, Hertfordshire (GB)

(73) Assignee: Glaxo Group Limited

(21) Appl. No.: 13/124,596

(22) PCT Filed: Oct. 16, 2009
(86) PCT No.: PCT/EP2009/063610

§ 371 Date: Apr. 15, 2011

Related U.S. Application Data
(60) Provisional application No. 61/106,279, filed on Oct. 17, 2008.

(51) Int. Cl. A61K 31/4375; A61P 31/04; C07D 471/16

(52) U.S. Cl. 514/292; 546/86

(57) ABSTRACT

The present invention relates to tricyclic nitrogen containing compounds of Formula (I):

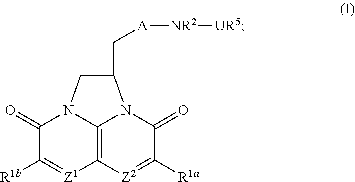

or
pharmaceutically acceptable salts and/or N-oxides thereof, corresponding pharmaceutical compositions, preparation and/or treatment methods for bacterial infections thereof.

This invention relates to novel compounds, compositions containing them and their use as antibacterials.

WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2006002047, WO2006014580, WO2006010040, WO2006017326, WO2006012396, WO2006017468, WO2006020561, WO2006081179, WO2006081264, WO2006081289, WO2006081178, WO2006081182, WO01/25227, WO02/40474, WO02/07572, WO2004024712, WO2004024713, WO2004035569, WO2004087647, WO2004089947, WO2005016916, WO2005097781, WO2006010831, WO2006021448, WO2006032466, WO2006038172, WO2006046552, WO06099884, WO06126171, WO06137485, WO06105289, WO06125974, WO06134378, WO07016610, WO07081597, WO07071936, WO07115947, WO07118130, WO07122258, WO08006648, WO08003690 and WO08009700 disclose quinoline, naphthyridine, morpholine, cyclohexane, piperidine and piperazine derivatives having antibacterial activity. WO2004104000 discloses tricyclic condensed ring compounds capable of selectively acting on cannabinoid receptors. WO2003048081, WO2003048158 and US2003232804 disclose glycinamides as Factor Xa inhibitors.

This invention provides a compound of formula (I) or a pharmaceutically acceptable salt and/or N-oxide thereof:

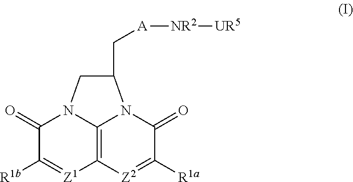

wherein:
one of Z¹ and Z² is CR^{1c} and the other is CH or N;
R^{1a} and R^{1b} are independently selected from hydrogen; halogen; cyano; (C₁₋₆)alkyl; (C₁₋₆)alkylthio; trifluoromethyl; trifluoromethoxy; carboxy; (C₁₋₆)alkoxy; hydroxy; hydroxy optionally substituted with (C₁₋₆)alkoxy-substituted(C₁₋₆)alkyl; (C₁₋₆)alkoxy-substituted(C₁₋₆)alkyl; hydroxy(C₁₋₆)alkyl; an amino group optionally N-substituted by one or two (C₁₋₆)alkyl, formyl, (C₁₋₆)alkylcarbonyl or (C₁₋₆)alkylsulphonyl groups; and aminocarbonyl wherein the amino group is optionally substituted by one or two (C₁₋₄)alkyl;
provided that R^{1a} and R^{1b} are H when Z² or Z¹ is N, respectively;
R^{1c} is (C₁₋₆)alkyl;
R² is hydrogen, (C₁₋₄)alkyl, or together with R⁶ forms Y as defined below;
A is a group (i) selected from:

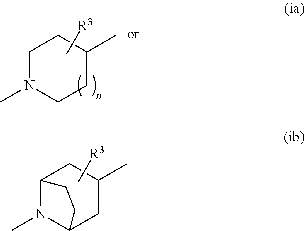

in which:

R³ is independently as defined for R^{1a} and R^{1b} or is oxo, and n is 1 or 2;

or A is a group (ii):

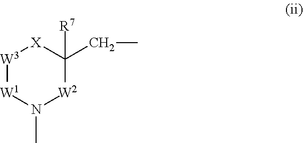

wherein:

W¹, W² and W³ are CR⁴R⁸,

or W² and W³ are CR⁴R⁸ and W¹ represents a bond between W³ and N;

X is O, CR⁴R⁸, or NR⁶;

one R⁴ is independently as defined for R^{1a} and R^{1b} and the remainder and R⁸ are hydrogen,

or one R⁴ and R⁸ are together oxo and the remainder are hydrogen;

R⁶ is hydrogen, (C₁₋₆)alkyl, or together with R² forms Y;

R⁷ is hydrogen; halogen; (C₁₋₆)alkoxy; hydroxy; or (C₁₋₆)alkyl;

Y is CR⁴R⁸CH₂; CH₂CR⁴R⁸; (C═O); CR⁴R⁸; CR⁴R⁸(C═O); or (C═O)CR⁴R⁸ where R⁴ and R⁸ are independently as defined above;

or when X is CR⁴R⁸, R⁸ and R⁷ together represent a bond;

U is selected from CO and CH₂; and
R⁵ is an optionally substituted bicyclic carbocyclic or heterocyclic ring system (B):

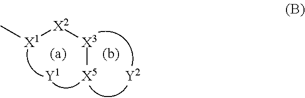

containing up to four heteroatoms in each ring in which

at least one of rings (a) and (b) is aromatic;

X¹ is C or N when part of an aromatic ring, or CR¹⁴ when part of a non-aromatic ring;

X² is N, NR¹³, O, S(O)_{X}, CO or CR¹⁴ when part of an aromatic or non-aromatic ring or may in addition be CR¹⁴R¹⁵ when part of a non aromatic ring;

X³ and X⁵ are independently N or C;

Y¹ is a 0 to 4 atom linker group each atom of which is independently selected from N, NR¹³, O, S(O)_{X}, CO and CR¹⁴ when part of an aromatic or non-aromatic ring or may additionally be CR¹⁴R¹⁵ when part of a non aromatic ring;

Y² is a 2 to 6 atom linker group, each atom of Y² being independently selected from N, NR¹³, O, S(O)_{X}, CO, CR¹⁴ when part of an aromatic or non-aromatic ring or may additionally be CR¹⁴R¹⁵ when part of a non aromatic ring;

each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or

R¹⁴ and R¹⁵ may together represent oxo;

each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl; and

each x is independently 0, 1 or 2.

This invention also provides a method of treatment of bacterial infections in mammals, particularly in man, which method comprises the administration to a mammal in need of such treatment an effective amount of a compound of formula (I), or a pharmaceutically acceptable salt and/or N-oxide thereof.

The invention also provides the use of a compound of formula (I), or a pharmaceutically acceptable salt and/or N-oxide thereof, in the manufacture of a medicament for use in the treatment of bacterial infections in mammals.

The invention also provides a pharmaceutical composition comprising a compound of formula (I), or a pharmaceutically acceptable salt and/or N-oxide thereof, and a pharmaceutically acceptable carrier.

In particular aspects:

Z¹ is CR^{1c} and Z² is CH;

Z¹ is CH and Z² is CR^{1c};

Z¹ is CR^{1c} and Z² is N; or

Z¹ is N and Z² is CR^{1c}.

In a particular aspects R^{1a} and R^{1b} are independently hydrogen, (C₁₋₄)alkoxy, (C₁₋₄)alkylthio, (C₁₋₄)alkyl, cyano, carboxy, hydroxymethyl or halogen; more particularly hydrogen, methoxy, methyl, cyano, or halogen.

In particular embodiments R^{1a} and R^{1b} are hydrogen.

In particular embodiments R^{1c} is methyl.

In a particular aspect R² is hydrogen.

In particular embodiments R³ is selected from hydrogen; (C₁₋₆)alkoxy; hydroxy; hydroxy optionally substituted with (C₁₋₆)alkoxy-substituted(C₁₋₆)alkyl; optionally substituted amino; halogen; (C₁₋₄) alkyl; 1-hydroxy-(C₁₋₄) alkyl; and optionally substituted aminocarbonyl. In more particular embodiments, R³ is selected from hydrogen; CONH₂; 1-hydroxyalkyl (e.g. CH₂OH); (C₁₋₆)alkoxy (e.g. methoxy); hydroxy; hydroxy optionally substituted with (C₁₋₆)alkoxy-substituted(C₁₋₆)alkyl; optionally substituted amino; and halogen (e.g. fluoro). Most particularly R³ is hydrogen, hydroxy or fluoro.

In a particular aspect, when A is (ia), n is 1. In a further aspect R³ is in the 3- or 4-position. In a more particular aspect, A is (ia), n is 1 and R³ is in the 3-position, and more particularly is cis to the NR² group. In particular embodiments, A is a group (ia) in which n is 1 and R³ is hydrogen or hydroxy. More particularly where A is 3-hydroxy-piperidin-4-yl the configuration is (3R,4S) or (3S,4R). Alternatively and more particularly where A is piperidin-4-yl the configuration is (3R,4S). In some embodiments, R³ is hydrogen.

In an alternative more particular aspect, when A is (ia), n is 1, R³ is in the 4-position and is methyl.

In a particular aspect, when A is (ii), X is CR⁴R⁸ and R⁸ is H and R⁴ is H or OH and more particularly OH is trans to R7. In a further aspect W¹ is a bond. In another aspect R⁷ is H. In an additional aspect W¹ is a bond, W² and W³ are both CH₂ and R⁷ is H. Where A is 4-hydroxypyrrolidin-3-ylmethyl, in a particular aspect the configuration is (3S,4S). Where A is pyrrolidin-3-ylmethyl, in a particular aspect the configuration is 3S.

In a particular aspect, when A is (ii), X is O, R⁷ is H and W¹, W² and W³ are each CH₂.

In certain embodiments U is CH₂.

In certain embodiments R⁵ is an aromatic heterocyclic ring (B) having 8-11 ring atoms including 2-4 heteroatoms of which at least one is N or NR¹³ in which, in particular embodiments, Y² contains 2-3 heteroatoms, one of which is S and 1-2 are N, with one N bonded to X³.

In alternative embodiments the heterocyclic ring (B) has ring (a) aromatic selected from optionally substituted benzo, pyrido, pyridazino, pyrimidino and pyrazino; and ring (b) non aromatic in which Y² has 3-5 atoms, more particularly 3 or 4 atoms, including at least one heteroatom, with O, S, CH₂ or NR¹³ bonded to X⁵ where R¹³ is hydrogen or other than hydrogen, and either NHCO bonded via N to X³, or O, S, CH₂ or NH bonded to X³. In a particular aspect the ring (a) contains aromatic nitrogen, and more particularly ring (a) is pyrido or pyrazino. Examples of rings (B) include optionally substituted:

### (a) and (b) Aromatic

1H-pyrrolo[2,3-b]-pyridin-2-yl, 1H-pyrrolo[3,2-b]-pyridin-2-yl, 3H-imidazo[4,5-b]-pyrid-2-yl, 3H-quinazolin-4-one-2-yl, benzimidazol-2-yl, benzo[1,2,3]-thiadiazol-5-yl, benzo[1,2,5]-oxadiazol-5-yl, benzofur-2-yl, benzothiazol-2-yl, benzo[b]thiophen-2-yl, benzoxazol-2-yl, chromen-4-one-3-yl, imidazo[1,2-a]pyridin-2-yl, imidazo-[1,2-a]-pyrimidin-2-yl, indol-2-yl, indol-6-yl, isoquinolin-3-yl, [1,8]-naphthyridine-3-yl, oxazolo[4,5-b]-pyridin-2-yl, quinolin-2-yl, quinolin-3-yl, quinoxalin-2-yl, naphthalen-2-yl, 1,3-dioxo-isoindol-2yl, 1H-benzotriazol-5-yl, 1H-indol-5-yl, 3H-benzooxazol-2-one-6-yl, 3H-benzooxazol-2-thione-6-yl, 3H-benzothiazol-2-one-5-yl, 3H-quinazolin-4-one-6-yl, pyrido[1,2-a]pyrimidin-4-one-3-yl (4-oxo-4H-pyrido[1,2-a]pyrimidin-3-yl), benzo[1,2,3]thiadiazol-6-yl, benzo[1,2,5]thiadiazol-5-yl, benzo[1,4]oxazin-2-one-3-yl, benzothiazol-5-yl, benzothiazol-6-yl, cinnolin-3-yl, imidazo[1,2-b]pyridazin-2-yl, pyrazolo[1,5-a]pyrazin-2-yl, pyrazolo[1,5-a]pyridin-2-yl, pyrazolo[1,5-a]pyrimidin-6-yl, pyrazolo[5,1-c][1,2,4]triazin-3-yl, pyrido[1,2-a]pyrimdin-4-one-2-yl(4-oxo-4H-pyrido[1,2-a]pyrimidin-2-yl), quinazolin-2-yl, quinoxalin-6-yl, thiazolo[3,2-a]pyrimidin-5-one-7-yl, thiazolo[5,4-b]pyridin-2-yl, thieno[3,2-b]pyridin-6-yl, thiazolo[5,4-b]pyridin-6-yl, thiazolo[4,5-b]pyridin-5-yl, [1,2,3]thiadiazolo[5,4-b]pyridin-6-yl, 2H-isoquinolin-1-one-3-yl (1-oxo-1,2-dihydro-isoquinolin-3-yl), [1,2,3]thiadiazolo[5,4-b]pyridine-6-yl

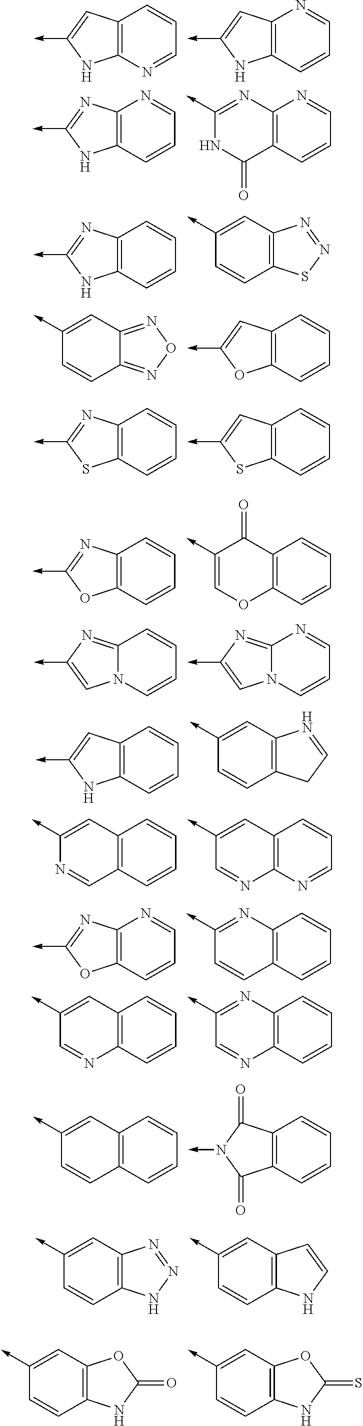

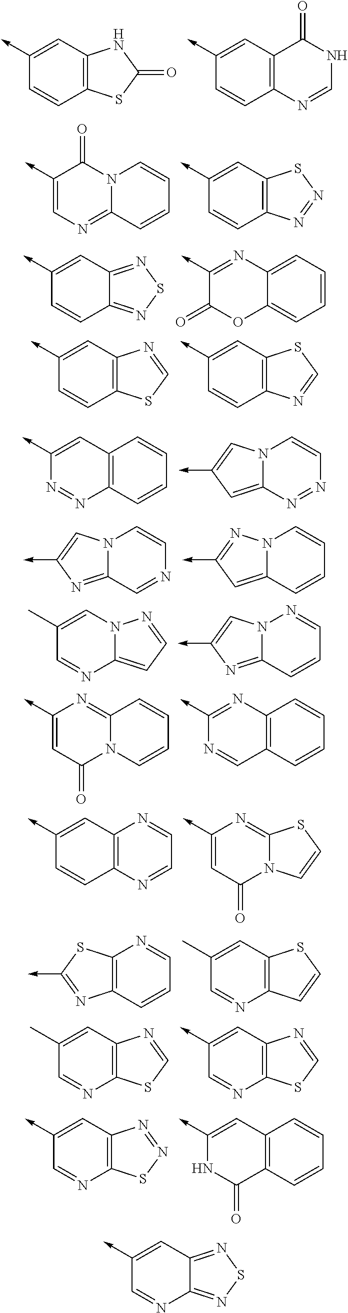

in which → is the point of attachment

### (a) is Non Aromatic

(2S)-2,3-dihydro-1H-indol-2-yl, (2S)-2,3-dihydro-benzo[1,4]dioxine-2-yl, 3-(R,S)-3,4-dihydro-2H-benzo[1,4]thiazin-3-yl, 3-(R)-2,3-dihydro-[1,4]dioxino[2,3-b]pyridin-3-yl, 3-(S)-2,3-dihydro-[1,4]dioxino[2,3-b]pyridin-3-yl, 2,3-dihydro-benzo[1,4]dioxan-2-yl, 3-substituted-3H-quinazolin-4-one-2-yl,

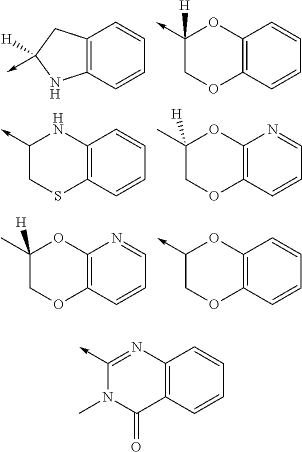

in which → is the point of attachment

### (b) is Non Aromatic

1,1,3-trioxo-1,2,3,4-tetrahydro-1 l⁶-benzo[1,4]thiazin-6-yl, benzo[1,3]dioxol-5-yl, 2,3-dihydro-benzo[1,4]dioxin-6-yl, 3-substituted-3H-benzooxazol-2-one-6-yl, 3-substituted-3H-benzooxazole-2-thione-6-yl, 3-substituted-3H-benzothiazol-2-one-6-yl, 4H-benzo[1,4]oxazin-3-one-6-yl(3-oxo-3,4-dihydro-2H-benzo[1,4]oxazin-6-yl), 4H-benzo[1,4]thiazin-3-one-6-yl (3-oxo-3,4-dihydro-2H-benzo[1,4]thiazin-6-yl), 4H-benzo[1,4]oxazin-3-one-7-yl, 4-oxo-2,3,4,5-tetrahydro-benzo[b][1,4]thiazepine-7-yl, 5-oxo-2,3-dihydro-5H-thiazolo[3,2-a]pyrimidin-6-yl, 1H-pyrido[2,3-b][1,4]thiazin-2-one-7-yl (2-oxo-2,3-dihydro-1H-pyrido[2,3-b]thiazin-7-yl), 2,3-dihydro-1H-pyrido[2,3-b][1,4]thiazin-7-yl, 2-oxo-2,3-dihydro-1H-pyrido[3,4-b]thiazin-7-yl, 2,3-dihydro-[1,4]dioxino[2,3-b]pyridin-6-yl, 2,3-dihydro-[1,4]dioxino[2,3-c]pyridin-7-yl, 2,3-dihydro-[1,4]dioxino[2,3-b]pyridin-7-yl, 3,4-dihydro-2H-benzo[1,4]oxazin-6-yl, 3,4-dihydro-2H-benzo[1,4]thiazin-6-yl, 3-oxo-3,4-dihydro-2H-pyrido[3,2-b][1,4]oxazin-6-yl, 3,4-dihydro-2H-pyrido[3,2-b][1,4]thiazin-6-yl, 3-oxo-3,4-dihydro-2H-pyrido[3,2-b][1,4]thiazin-6-yl, 3,4-dihydro-1H-quinolin-2-one-7-yl, 3,4-dihydro-1H-quinoxalin-2-one-7-yl, 6,7-dihydro-4H-pyrazolo[1,5-a]pyrimidin-5-one-2-yl, 5,6,7,8-tetrahydro-[1,8]naphthyridin-2-yl(1,2,3,4-tetrahydro-[1,8]naphthyridin-7-yl), 2-oxo-3,4-dihydro-1H-[1,8]naphthyridin-6-yl, 6-oxo-6,7-dihydro-5H-pyridazino[3,4-b][1,4]thiazin-3-yl(6-oxo-6,7-dihydro-5H-8-thia-1,2,5-triaza-naphthalen-3-yl), 2-oxo-2,3-dihydro-1H-pyrido[3,4-b][1,4]oxazin-7-yl, 2-oxo-2,3-dihydro-1H-pyrido[2,3-b][1,4]oxazin-7-yl, 6,7-dihydro-[1,4]dioxino[2,3-d]pyrimidin-2-yl, [1,3]oxathiolo[5,4-c]pyridin-6-yl, 3,4-dihydro-2H-pyrano[2,3-c]pyridin-6-yl, 2,3-dihydro[1,4]oxathiino[2,3-c]pyridin-7-yl, 6,7-dihydro[1,4]dioxino[2,3-c]pyridazin-3-yl, 5,6,7,8-tetrahydroisoquinolin-3-yl, 6,7-dihydro-5H-cyclopenta[c]pyridin-3-yl, 1,3-dihydrofuro[3,4-c]pyridin-6-yl, 3,4-dihydro-2H-[1,4]oxathiepino[2,3-c]pyridin-8-yl, [1,3]oxathiolo[4,5-c]pyridin-6-yl, 6,7-dihydro[1,4]oxathiino[2,3-c]pyridazin-3-yl, 6,7-dihydro-5H-pyrano[2,3-c]pyridazin-3-yl, 5,6-dihydrofuro[2,3-c]pyridazin-3-yl, 2,3-dihydrofuro[2,3-c]pyridin-5-yl, 2-substituted 1H-pyrimido[5,4-b][1,4]oxazin-7(6H)-one, 2-substituted 5,6-dihydropyrido[2,3-d]pyrimidin-7(1H)-one, 7-substituted 2H-chromen-2-one, 7-substituted 2H-pyrano[2,3-b]pyridin-2-one, 2-substituted 6,7-dihydro-5H-pyrano[2,3-d]pyrimidine, 8-substitited 2H-pyrido[1,2-a]pyrimidin-2-one, 2,3-dihydro-1-benzofuran-5-yl, 1H-pyrimido[5,4-b][1,4]thiazin-7(6H)-one-2-yl, 3,4-dihydro-2H-chromen-7-yl, 2,3-dihydro-1-benzofuran-6-yl, 3,4-dihydro-2H-chromen-6-yl, 3,4-dihydro-2H-pyrido[3,2-b][1,4]oxazine-6-yl, 3,4-dihydro-2H-pyrido[3,2-b][1,4]thiazine-6-yl, 6,7-dihydro-5H-thieno[3,2-b]pyran-2-yl, 2,3,4,5-tetrahydro-1,5-benzothiazepine-7-yl.

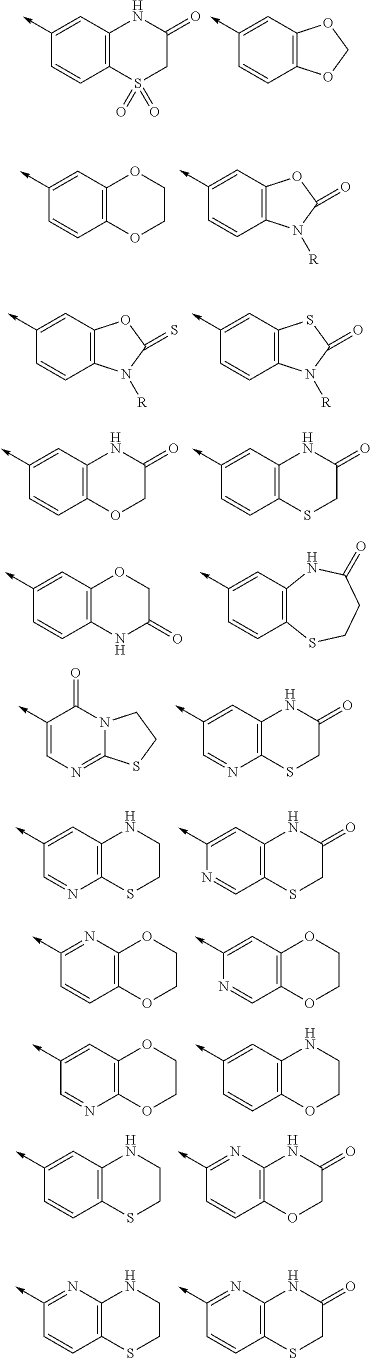

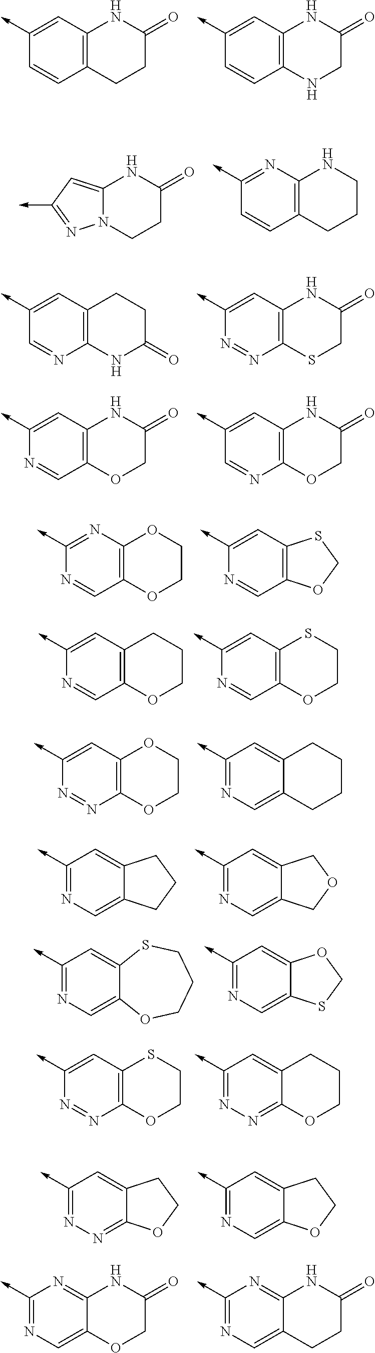

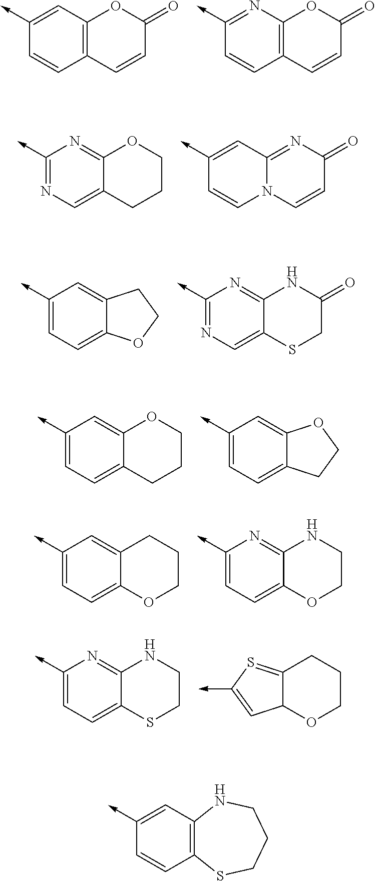

where R is an optional substituent (such as R¹³ as defined herein) and → is the point of attachment.

In some embodiments R¹³ is H if in ring (a) or in addition (C₁₋₄)alkyl such as methyl or isopropyl when in ring (b). More particularly, in ring (b) R¹³ is H when NR¹³ is bonded to X³ and (C₁₋₄)alkyl when NR¹³ is bonded to X⁵.

In further embodiments R¹⁴ and R¹⁵ are independently selected from hydrogen, halo, hydroxy, (C₁₋₄) alkyl, (C₁₋₄)alkoxy, nitro and cyano. More particularly R¹⁵ is hydrogen.

More particularly each R¹⁴ is independently selected from hydrogen, chloro, fluoro, hydroxy, methyl, methoxy, nitro and cyano. Still more particularly R¹⁴ is independently selected from hydrogen, fluorine and nitro.

Most particularly R¹⁴ and R¹⁵ are each H.

Particular groups R⁵ include:
[1,2,3]thiadiazolo[5,4-b]pyridin-6-yl
1H-pyrrolo[2,3-b]pyridin-2-yl
2,3-dihydro-[1,4]dioxino[2,3-b]pyridin-6-yl
2,3-dihydro-[1,4]dioxino[2,3-b]pyridin-7-yl
2,3-dihydro-[1,4]dioxino[2,3-c]pyridin-7-yl
2,3-dihydro-benzo[1,4]dioxin-6-yl
2-oxo-2,3-dihydro-1H-pyrido[2,3-b][1,4]oxazin-7-yl
2-oxo-2,3-dihydro-1H-pyrido[2,3-b][1,4]thiazin-7-yl
3,4-dihydro-2H-benzo[1,4]oxazin-6-yl
3-methyl-2-oxo-2,3-dihydro-benzooxazol-6-yl
3-oxo-3,4-dihydro-2H-benzo[1,4]oxazin-6-yl
3-oxo-3,4-dihydro-2H-pyrido[3,2-b][1,4]oxazin-6-yl
3-oxo-3,4-dihydro-2H-benzo[1,4]thiazin-6-yl (4H-benzo[1,4]thiazin-3-one-6-yl)
4-oxo-4H-pyrido[1,2-a]pyrimidin-2-yl
6-nitro-benzo[1,3]dioxol-5-yl
7-fluoro-3-oxo-3,4-dihydro-2H-benzo[1,4]oxazin-6-yl
8-hydroxy-1-oxo-1,2-dihydro-isoquinolin-3-yl
8-hydroxyquinolin-2-yl
benzo[1,2,3]thiadiazol-5-yl
benzo[1,2,5]thiadiazol-5-yl
benzothiazol-5-yl
thiazolo-[5,4-b]pyridin-6-yl
3-oxo-3,4-dihydro-2H-pyrido[3,2-b][1,4]thiazin-6-yl
7-chloro-3-oxo-3,4-dihydro-2H-pyrido[3,2-b][1,4]thiazin-6-yl
7-chloro-3-oxo-3,4-dihydro-2H-pyrido[3,2-b][1,4]oxazin-6-yl
7-fluoro-3-oxo-3,4-dihydro-2H-pyrido[3,2-b][1,4]thiazin-6-yl
2-oxo-2,3-dihydro-1H-pyrido[3,4-b][1,4]thiazin-7-yl
[1,3]oxathiolo[5,4-c]pyridin-6-yl
3,4-dihydro-2H-pyrano[2,3-c]pyridin-6-yl
5-carbonitro-2,3-dihydro-1,4-benzodioxin-7-yl
2,3-dihydro[1,4]oxathiino[2,3-c]pyridin-7-yl
6,7-dihydro[1,4]dioxino[2,3-c]pyridazin-3-yl
5,6,7,8-tetrahydroisoquinolin-3-yl
6,7-dihydro-5H-cyclopenta[c]pyridin-3-yl
1,3-dihydrofuro[3,4-c]pyridin-6-yl
6-fluoro-2,3-dihydro-1,4-benzodioxin-7-yl
3,4-dihydro-2H-[1,4]oxathiepino[2,3-c]pyridin-8-yl,
[1,3]oxathiolo[4,5-c]pyridine-6-yl
2,3-dihydro-1-benzofuran-5-yl
6,7-dihydro[1,4]oxathiino[2,3-c]pyridazin-3-yl
6,7-dihydro-5H-pyrano[2,3-c]pyridazin-3-yl
5,6-dihydrofuro[2,3-c]pyridazin-3-yl
2-substituted 1H-pyrimido[5,4-b][1,4]oxazin-7(6H)-one
2-substituted 4-chloro-1H-pyrimido[5,4-b][1,4]oxazin-7(6H)-one
2-substituted 5,6-dihydropyrido[2,3-d]pyrimidin-7(1H)-one
2-substituted 4-chloro-5,6-dihydropyrido[2,3-d]pyrimidin-7(1H)-one
2-substituted 4-methyl-5,6-dihydropyrido[2,3-d]pyrimidin-7(1H)-one
2-substituted 4-methyloxy-5,6-dihydropyrido[2,3-d]pyrimidin-7(1H)-one
7-substituted 2H-chromen-2-one
7-substituted 2H-pyrano[2,3-b]pyridin-2-one
4-chloro-6,7-dihydro-5H-pyrano[2,3-c]pyrimidin-2-yl
8-substituted 2H-pyrido[1,2-a]pyrimidin-2-one
6,7-dihydro-5H-pyrano[2,3-c]pyrimidin-2-yl)
5-chloro-1-benzothiophen-2-yl
6-chloro-1-benzothiophen-2-yl
1-benzothiophen-5-yl
1-methyl-1H-1,2,3-benzotriazol-6-yl
imidazo[2,1-b][1,3]thiazol-6-yl
4-methyl-3,4-dihydro-2H-1,4-benzoxazin-7-yl
1-methyl-1H-indol-2-yl
1H-pyrimido[5,4-b][1,4]thiazin-7(6H)-one-2-yl
[1,2,5]thiadiazolo[3,4-b]pyridine-6-yl
4-fluoro-1H-benzimidazol-2-yl
3,4-dihydro-2H-chromen-7-yl
2,3-dihydro-1-benzofuran-6-yl
3,4-dihydro-2H-chromen-6-yl
6-chloro-2,3-dihydro-1,4-benzodioxin-7-yl
7-chloro-3,4-dihydro-2H-pyrido[3,2-b][1,4]oxazine-6-yl
7-chloro-3,4-dihydro-2H-pyrido[3,2-b][1,4]thiazine-6-yl
3,4-dihydro-2H-pyrido[3,2-b][1,4]thiazin-6-yl
5-fluoro-2,3-dihydro-1,4-benzodioxin-7-yl
5-fluoro-2,3-dihydro-1,4-benzodioxin-6-yl
8-fluoro-2H-1,4-benzoxazin-3(4H)-one-6-yl
8-fluoro-3,4-dihydro-2H-1,4-benzoxazin-6-yl
7,8-difluoro-3,4-dihydro-2H-1,4-benzoxazin-6-yl
6,7-dihydro-5H-thieno[3,2-b]pyran-2-yl
5-methyl-2,3-dihydro-1,4-benzodioxin-7-yl
4-oxo-2,3,4,5-tetrahydro-1,5-benzothiazepin-7-yl
3,4-dihydro-2H-1,4-benzothiazine-6-yl
2,3,4,5-tetrahydro-1,5-benzothiazepine-7-yl
7-fluoro-3,4-dihydro-2H-1,4-benzoxazine-6-yl

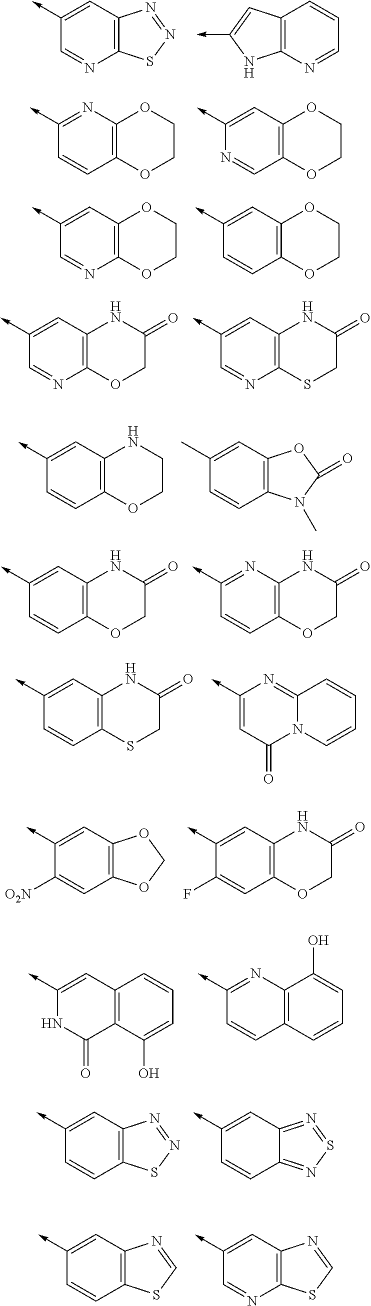

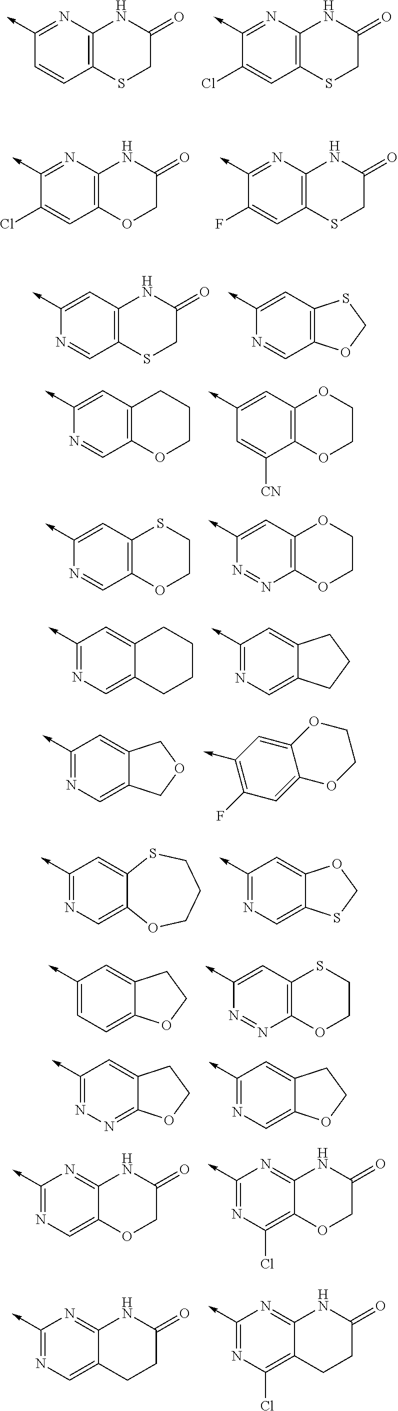

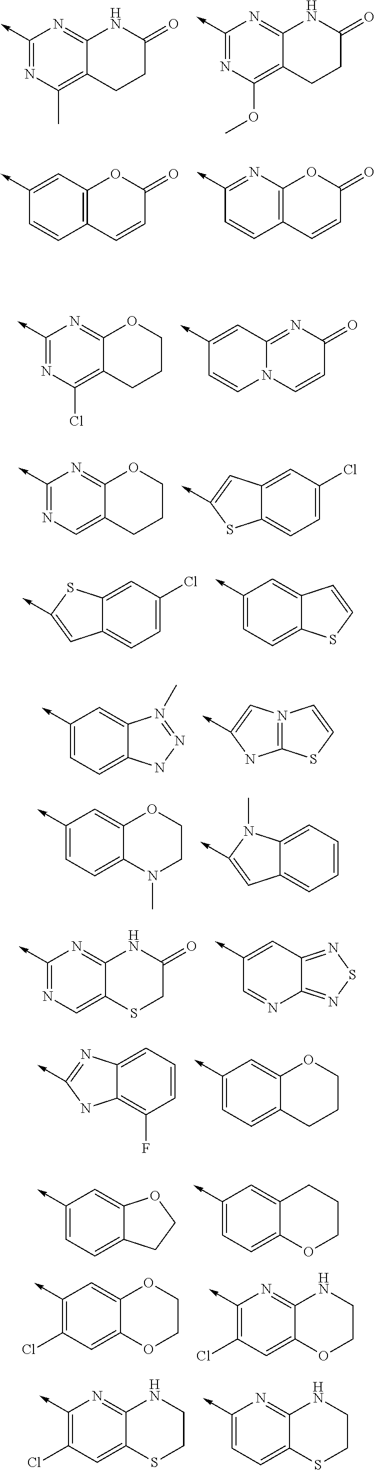

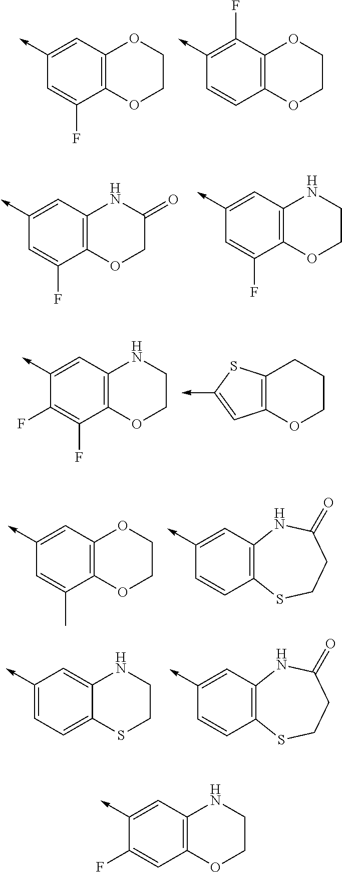

in which → is the point of attachment;
especially
3-oxo-3,4-dihydro-2H-pyrido[3,2-b][1,4]oxazin-6-yl
3-oxo-3,4-dihydro-2H-pyrido[3,2-b][1,4]thiazin-6-yl
2,3-dihydro-[1,4]dioxino[2,3-c]pyridin-7-yl
[1,3]oxathiolo[5,4-c]pyridin-6-yl
6-fluoro-2,3-dihydro-1,4-benzodioxin-7-yl
2,3-dihydro[1,4]oxathiino[2,3-c]pyridin-7-yl
3,4-dihydro-2H-pyrano[2,3-c]pyridin-6-yl
5-fluoro-2,3-dihydro-1,4-benzodioxin-7-yl
5-carbonitro-2,3-dihydro-1,4-benzodioxin-7-yl
2,3-dihydro-benzo[1,4]dioxin-6-yl

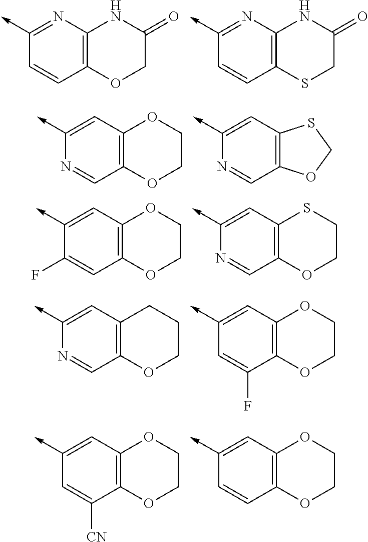

in which → is the point of attachment.

In some embodiments, R⁵ is:
2,3-dihydro-[1,4]dioxino[2,3-c]pyridin-7-yl;
[1,3]oxathiolo[5,4-c]pyridin-6-yl;
2,3-dihydro[1,4]oxathiino[2,3-c]pyridin-7-yl; or
3,4-dihydro-2H-pyrano[2,3-c]pyridin-6-yl.

In some embodiments, Z¹ is CR^{1c}, R^{1c} is methyl, Z² is CH, R^{1a} and R^{1b} are hydrogen, A is (ia) wherein n is 1 and R³ is hydrogen, R² is hydrogen, U is CH₂ and R⁵ is any of the embodiments defined above, including individual embodiments.

Particular examples of compounds of the invention include:
(1R)-1-({4-[(3,4-dihydro-2H-pyrano[2,3-c]pyridin-6-ylmethyl)amino]-1-piperidinyl}methyl)-6-methyl-1,2-dihydro-4H,9H-imidazo[1,2,3-ij]-1,8-naphthyridine-4,9-dione;
(1R)-1-({4-[(2,3-dihydro[1,4]oxathiino[2,3-c]pyridin-7-ylmethyl)amino]-1-piperidinyl}methyl)-6-methyl-1,2-dihydro-4H,9H-imidazo[1,2,3-ij]-1,8-naphthyridine-4,9-dione;
(1R)-1-({4-[(2,3-dihydro[1,4]dioxino[2,3-c]pyridin-7-ylmethyl)amino]-1-piperidinyl}methyl)-6-methyl-1,2-dihydro-4H,9H-imidazo[1,2,3-ij]-1,8-naphthyridine-4,9-dione; and
(1R)-6-methyl-1-({4-[([1,3]oxathiolo[5,4-c]pyridin-6-ylmethyl)amino]-1-piperidinyl}methyl)-1,2-dihydro-4H,9H-imidazo[1,2,3-ij]-1,8-naphthyridine-4,9-dione;
   and pharmaceutically acceptable salts and/or N-oxides thereof.

When used herein, the terms “alkyl”, “alkenyl”, and “alkoxy” include groups having straight and branched chains.

When a range of carbon numbers is used herein, e.g. “(C₁₋₆)”, each embodiment in the range having a particular number of carbon atoms (including a single integer or a sub-range of carbons, e.g. “(C₁₋₄)”, “(C₂₋₆)” etc.), is specifically included as though expressly set forth. For instance, (C₁₋₆)alkyl includes alkyl groups having 1, 2, 3, 4, 5 or 6 carbons, as well as (C₁₋₅), (C₂₋₆), (C₃₋₅), etc. alkyl. Thus (C₁₋₆)alkyl includes all straight and branched chain alkyl groups having from 1-6 carbons, e.g. methyl, ethyl, n-propyl, iso-propyl, n-butyl, iso-butyl, sec-butyl, t-butyl, pentyl and hexyl. The term ‘alkenyl’ should be interpreted accordingly.

Halo or halogen includes fluoro, chloro, bromo and iodo.

Haloalkyl moieties include 1-3 halogen atoms.

When a term includes “substituted alkyl,” e.g. “(C₁₋₆)alkoxy-substituted(C₁₋₆)alkyl”, the number of indicated substituents may be as permitted by valency and chemistry. In some embodiments, one or two (particularly one) of such substituents is present.

Also, when a term includes “substituted alkyl” or would evidently include substitution of an alkyl group (e.g., “carboxy(C₁₋₄)alkyl” and “hydroxy optionally substituted with (C₁₋₆)alkoxy-substituted(C₁₋₆)alkyl”), the substituent(s) may be present on any carbon atom of the alkyl group as permitted by chemistry. In some embodiments, the alkyl group is substituted in the 1-position.

Compounds within the invention contain a heterocyclyl group and may occur in two or more tautomeric forms depending on the nature of the heterocyclyl group; all such tautomeric forms are included within the scope of the invention.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Furthermore, it will be understood that phrases such as “a compound of formula (I) or a pharmaceutically acceptable salt and/or N-oxide thereof” are intended to encompass the compound of formula (I), an N-oxide of the compound of formula (I), a pharmaceutically acceptable salt of the compound of formula (I) or any pharmaceutically acceptable combination of these. As will be understood by those skilled in the art, such compounds may be in the form of a solvate. Thus by way of non-limiting example used here for illustrative purpose, “a compound of formula (I) or a pharmaceutically acceptable salt thereof” may include a pharmaceutically acceptable salt of a compound of formula (I) that is further present as a solvate.

Since the compounds of formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10% of a compound of the formula (I) or pharmaceutically acceptable salt and/or N-oxide thereof.

Particular compounds according to the invention include those mentioned in the examples and their pharmaceutically acceptable salts and/or N-oxides.

Pharmaceutically acceptable salts of the above-mentioned compounds of formula (I) include the acid addition or quaternary ammonium salts, for example their salts with mineral acids e.g. hydrochloric, hydrobromic, sulphuric, nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. The invention extends to all such derivatives.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. For example the invention includes enantiomers and diastereoisomers at the attachment point of NR² and R³. The different isomeric forms may be separated or resolved one from the other by conventional methods, or any given isomer may be obtained by conventional synthetic methods or by stereospecific or asymmetric syntheses. The invention also includes compounds of formula I in any polymorphic forms.

In a further aspect of the invention there is provided a process for preparing compounds of formula (I) where Z¹ is CR^{1c} and Z² is CH, and pharmaceutically acceptable salts and/or N-oxides thereof, which process comprises:

reacting a compound of formula (IIA):

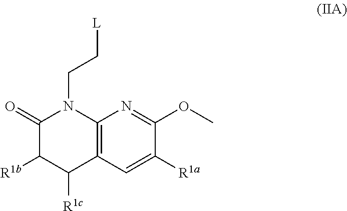

in which:
L is an epoxide or -A(Q¹)(Q²), where Q¹ and Q² are both attached to the same carbon atom on A, Q¹ is H and Q² is N(R²⁰)R^{2′} or Q¹ and Q² together form ethylenedioxy or oxo, R²⁰ is UR⁵ or a group convertible thereto and R^{2′} is R² or a group convertible thereto, and A, R^{1a}, R^{1b}, R^{1c}, R², U and R⁵, are as defined in formula (I), followed by cyclisation and oxidation, to give a compound of formula (IIIA):

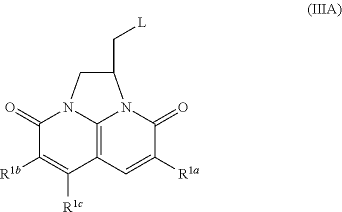

and thereafter optionally or as necessary converting L to -A-NR²—UR⁵, interconverting any variable groups, and/or forming a pharmaceutically acceptable salt and/or N-oxide thereof.

An example of the preparation of compounds of formula (IIIA) comprising reacting a compound of formula (IIA) is shown in Scheme 1 starting from commercially available reagents:

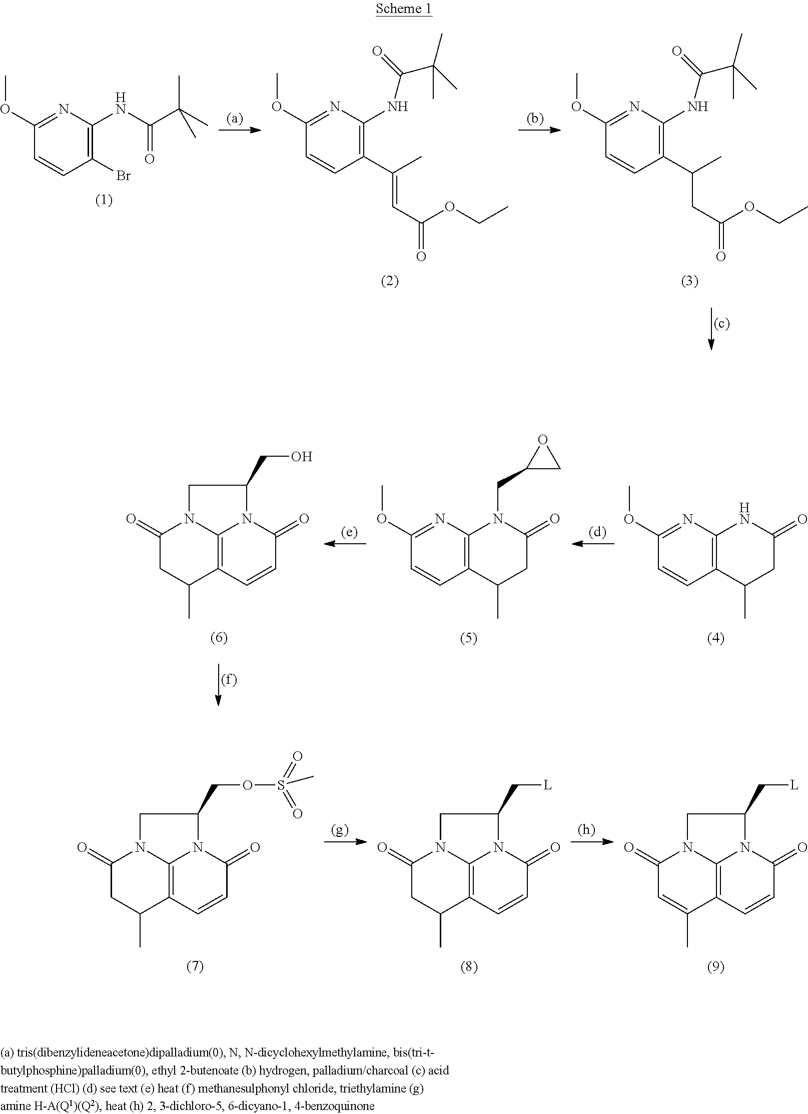

The reaction of known methoxypyridyl bromide (1) with ethyl 2-butenoate under standard Heck reaction conditions employing palladium catalysis (see Sydorenko, N, et al, Organic & Biomolecular Chemistry (2005), 3(11), 2140-2144 for an example of this type of catalysis in a Heck reaction) gives acryate (2). A mixture of olefins is possible; however, hydrogenation of the double bond of (2) will yield a single saturated product (3). Acid treatment of (3) to remove the pivalate residue and affect lactamisation yields the bicyclic lactam (4).

Conversion to the epoxide (5) can be effected in a number of ways. Reaction with epichlorohydrin under basic conditions affords racemic epoxide. Reaction with (commercially available) R or S-glycidyl nosylate ((2R)- or (2S)-2-oxiranylmethyl 3-nitrobenzenesulfonate) or (2R)- or (2S)-2-oxiranylmethyl 4-methylbenzenesulfonate, with base, e.g. sodium hydride or potassium t-butoxide, gives the corresponding chiral epoxides. Alternatively, allylation with allyl bromide under basic conditions affords the corresponding N-allyl material which can be epoxidised under standard achiral or chiral conditions to give the corresponding achiral or chiral epoxides.

Under thermal conditions, the epoxide (5) may be opened and cyclised directly to afford (6) (L=OH) which can be converted to the mesylate (7) (L=OMs). Displacement of the mesylate with amine H-A(Q¹)(Q²) such as 1,1-dimethylethyl 4-piperidinylcarbamate by heating in DMF affords (8). Oxidation to (9) may be carried out by oxidation of (8) with 2,3-dichloro-5,6-dicyano-1,4-benzoquinone (DDQ).

Subsequent conversion to compounds of formula (I) may be carried out as generally described herein. In particular, conversion of L to A(Q¹)(Q²) may be carried out on (7) or (9). As a further variation to Scheme 1, epoxide (5) may be prepared from (2) by first introducing a suitable epoxide precursor group (—CH₂—CHOH—CH₂OH, protected as a cyclic ester) before carrying out the steps (b) and (c).

As shown in Scheme 1 L in (IIIA) is not necessarily the same as L in (IIA). As will be appreciated by those skilled in the art, L in (IIA) is used to effect cyclization to the tricyclic (IIIA). The resulting “L” can be modified to attach the remaining portion of compound. This applies in analogous manner to the Schemes 2-4.

L may be a hydroxy group which can be oxidised to the aldehyde by conventional means such as 1,1,1-tris-(acetyloxy)-1,1-dihydro-1,2-benziodooxol-3-(1H)-one for reductive alkylation with HA-N(R²⁰)R^{2′} under conventional conditions (see for examples Smith, M. B.; March, J. M. Advanced Organic Chemistry, Wiley-Interscience 2001).

Alternatively L may be bromo which can be alkylated with HA-N(R²⁰)R^{2′} under conventional conditions.

Where Q1 and Q2 together form ethylenedioxy in formula (IIIA) the ketal may be converted to the ketone (Q¹ and Q² together form oxo) by conventional acid hydrolysis treatment with e.g. aqueous HCl or trifluoroacetic acid and the subsequent conversion to NR²UR⁵ by conventional reductive alkylation with amine NHR^{2′} R²⁰ (see for example Nudelman, A., et al, Tetrahedron 60 (2004) 1731-1748) and subsequent conversion to the required substituted amine, or directly with NHR²UR⁵, such as with sodium triacetoxyborohydride in dichloromethane/methanol.

Conveniently one of R²⁰ and R^{2′} is an N-protecting group, such as such as t-butoxycarbonyl, benzyloxycarbonyl or 9-fluorenylmethyloxycarbonyl. This may be removed by several methods well known to those skilled in the art (for examples see “Protective Groups in Organic Synthesis, T. W. Greene and P. G. M. Wuts, Wiley-Interscience, 1999), for example conventional acid hydrolysis with, for example trifluoroacetic acid or hydrochloric acid. The invention further provides compounds of formula (IIIA) in which L is -A-N(R²⁰)R^{2′} and R²⁰ is hydrogen.

The free amine of formula (IIIA) in which R²⁰ is hydrogen may be converted to NR²UR⁵ by conventional means such as amide formation with an acyl derivative R⁵COW, for compounds where U is CO or, where U is CH₂, by alkylation with an alkyl halide R⁵CH₂-halide in the presence of base, acylation/reduction with an acyl derivative R⁵COW or reductive alkylation with an aldehyde R⁵CHO under conventional conditions (see for examples Smith, M. B.; March, J. M. Advanced Organic Chemistry, Wiley-Interscience 2001). The appropriate reagents containing the required R⁵ group are known compounds or may be prepared analogously to known compounds, see for example WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO06002047, WO06014580, WO06010040, WO06017326, WO06012396, WO06017468, WO06020561, WO2004/035569, WO2004/089947, WO2003082835, WO06002047, WO06014580, WO06010040, WO06017326, WO06012396, WO06017468, WO06020561, WO06132739, WO06134378, WO06137485, WO06081179, WO06081264, WO06081289, WO06081178, WO06081182, WO07016610, WO07081597, WO07071936, WO07115947, WO07118130, WO07122258, WO08006648, WO08003690, WO08009700, WO2007067511 and EP0559285, each incorporated by reference herein in their entirety.

Where R⁵ contains an NH group, this may be protected with a suitable N-protecting group such as t-butoxycarbonyl, benzyloxycarbonyl or 9-fluorenylmethyloxycarbonyl during the coupling of the R⁵ derivative with the free amine of formula (IIB). The protecting group may be removed by conventional methods, such as by treatment with trifluoroacetic acid.

In a further aspect of the invention there is provided a process for preparing compounds of formula (I) where Z² is CR^{1c} and Z¹ is CH, and pharmaceutically acceptable salts and/or N-oxides thereof, which process comprises: reacting a compound of formula (IIB):

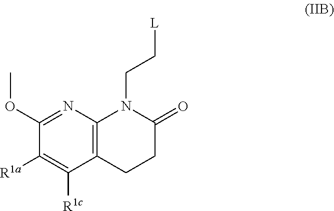

in which:
L is a leaving group or -A(Q¹)(Q²), where Q¹ and Q² are both attached to the same carbon atom on A, Q¹ is H and Q² is N(R²⁰)R^{2′} or Q¹ and Q² together form ethylenedioxy or oxo, R²⁰ is UR⁵ or a group convertible thereto and R^{2′} is R² or a group convertible thereto, and A, R^{1a}, R^{1c}, R², U and R⁵, are as defined in formula (I), followed by cyclisation and oxidation, to give a compound of formula formula (IIIB):

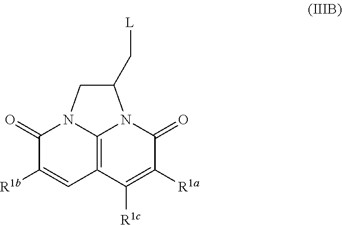

and thereafter optionally or as necessary converting L to -A-NR²—UR⁵, interconverting any variable groups, and/or forming a pharmaceutically acceptable salt and/or N-oxide thereof.

The reaction of (IIB) and subsequent transformations to form (IIIB) are carried out as for the preparation of compounds of formula (IIIA).

The invention further provides compounds of formula (IIIB) in which L is -A-N(R²⁰)R^{2′} and R²⁰ is hydrogen.

Compounds of formula (IIIB) in which L=-A(Q¹)(Q²) may be prepared by Scheme 2:

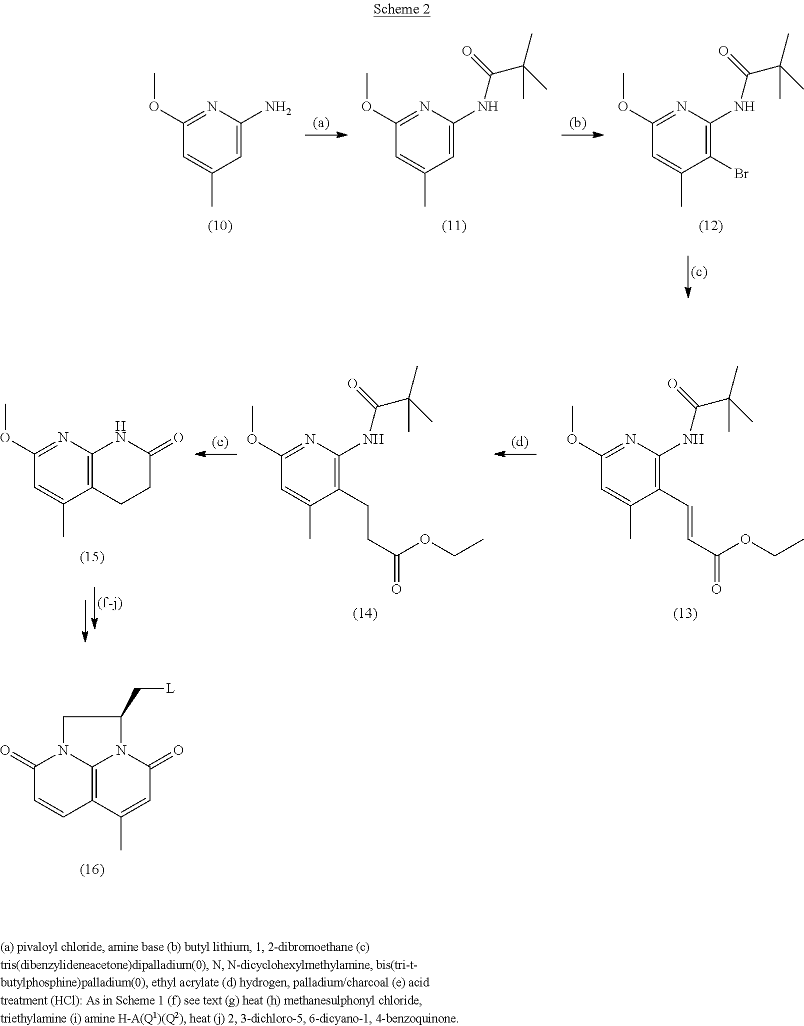

The commercially available 2-amino-4-methyl-6-methoxypyridine (10) may be acylated with pivaloyl chloride and an amine base such as triethylamine to give the acylated compound (II). Directed lithiation followed by lithium-halogen exchange with 1,2-dibromoethane gives brominated compound (12) (see for example Cottineau, et al, Tetrahedron 63 (2007) 10354-10362). The reaction of bromide (12) with ethyl acrylate under standard Heck reaction conditions employing palladium catalysis (see Sydorenko, N, et al, Organic & Biomolecular Chemistry (2005), 3(11), 2140-2144 for an example of this type of catalysis in a Heck reaction) gives olefin (13) which can be reduced under standard hydrogenation conditions to yield (14). Acid treatment of (14) to remove the pivalate residue and affect lactamisation yields the bicyclic lactam (15). The same series of reactions employed in Scheme 1 (see accompanied notes) can be utilized for the conversion of (15) to (IIIB) (16).

In a further aspect of the invention there is provided a process for preparing compounds of formula (I) where Z² is N and Z¹ is CR^{1c}, and pharmaceutically acceptable salts and/or N-oxides thereof, which process comprises:

reacting a compound of formula (IIC):

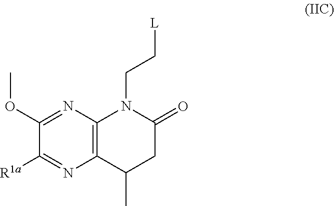

in which:
L is a leaving group or -A(Q¹)(Q²), where Q¹ and Q² are both attached to the same carbon atom on A, Q¹ is H and Q² is N(R²⁰)R^{2′} or Q¹ and Q² together form ethylenedioxy or oxo, R²⁰ is UR⁵ or a group convertible thereto and R^{2′} is R² or a group convertible thereto, and A, R^{1a}, R², U and R⁵ are as defined in formula (I), followed by cyclisation and oxidation to give a compound of formula (IIIC):

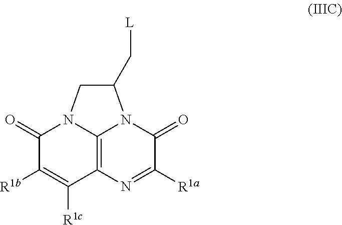

and thereafter optionally or as necessary converting L to -A-NR²—UR⁵, interconverting any variable groups, and/or forming a pharmaceutically acceptable salt and/or N-oxide thereof.

The reaction of (IIC) and subsequent transformations is carried out as for the preparation of compounds of formula (IIIB).

The invention further provides compounds of formula (IIIC) in which L is -A-N(R²⁰)R^{2′} and R²⁰ is hydrogen.

Compounds of formula (IIIC) in which L=-A(Q¹)(Q²) may be prepared by Scheme 3:

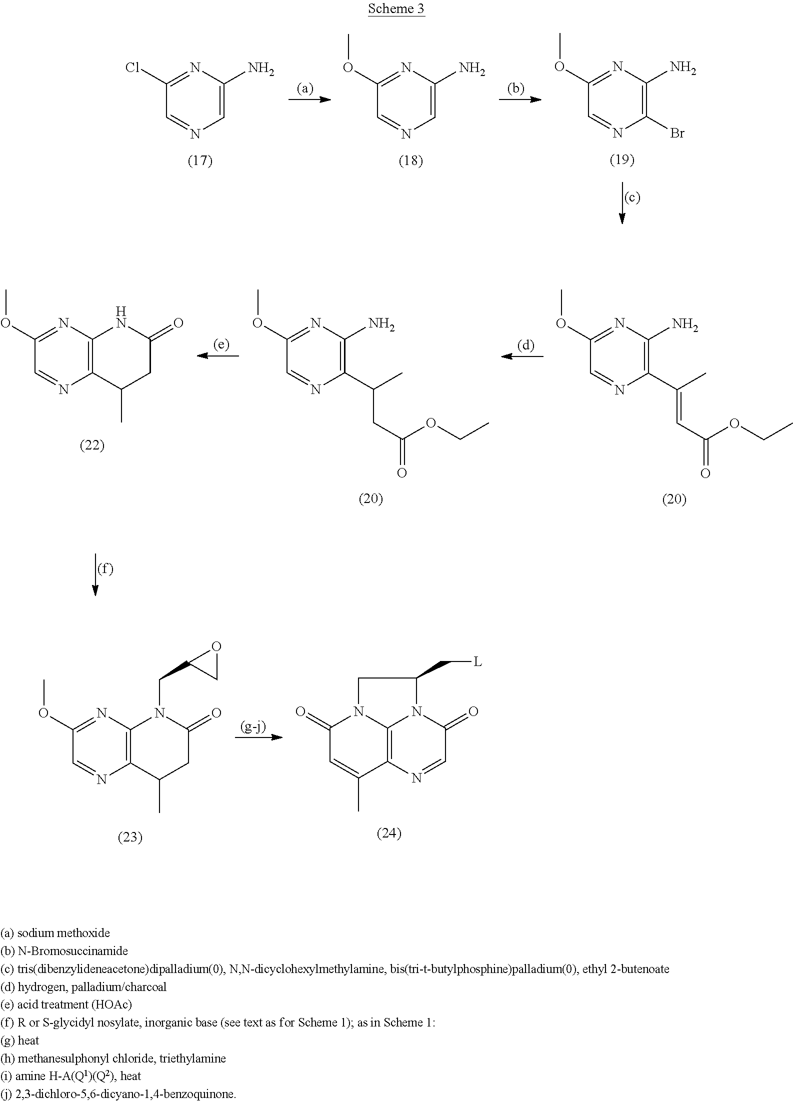

The commercially available 2-amino-6-chloropyrazine (17) can undergo a nucleophilic displacement with sodium methoxide to give (18). Bromination with NBS gives predominately 2-Amino-3-bromo-6-methoxy-pyrazine (19) (see for related example Barlaam, B., et al, Bioorg. & Med. Chem. Lett. 15 (2005) 5446-5449). The reaction of bromide (19) with ethyl 2-butenoate under standard Heck reaction conditions employing palladium catalysis (see Sydorenko, N, et al, Organic & Biomolecular Chemistry (2005), 3(11), 2140-2144 for an example of this type of catalysis in a Heck reaction) gives acryate (20). A mixture of olefins is possible; however, hydrogenation of the double bond of (20) will yield a single saturated product (21). Mild acid treatment of (21) to affect lactamisation yields the bicyclic lactam (22).

The same series of reactions employed in Scheme 1 (see text) can be utilized for the conversion to (IIIC) (24).

In a further aspect of the invention there is provided a process for preparing compounds of formula (I) where Z² is CR^{1c} and Z¹ is N, and pharmaceutically acceptable salts and/or N-oxides thereof, which process comprises:

reacting a compound of formula (IID):

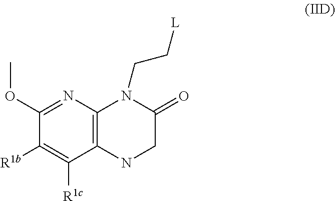

in which L is a leaving group or -A(Q¹)(Q²), where Q¹ and Q² are both attached to the same carbon atom on A, Q¹ is H and Q² is N(R²⁰)R^{2′} or Q¹ and Q² together form ethylenedioxy or oxo, R²⁰ is UR⁵ or a group convertible thereto and R^{2′} is R² or a group convertible thereto, and A, R^{1b}, R^{1c}, R², U and R⁵ are as defined in formula (I), followed by cyclisation and oxidation to give a compound of formula (IIID):

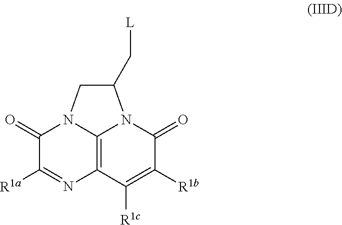

and thereafter optionally or as necessary converting L to -A-NR²—UR⁵, interconverting any variable groups, and/or forming a pharmaceutically acceptable salt and/or N-oxide thereof.

The reaction of (IID) and subsequent transformations are carried out as for the preparation of compounds of formula (IIIC).

The invention further provides compounds of formula (IIID) in which L is -A-N(R²⁰)R^{2′} and R²⁰ is hydrogen.

Compounds of formula (IIID) in which L=-A(Q¹)(Q²) may be prepared by Scheme 4:

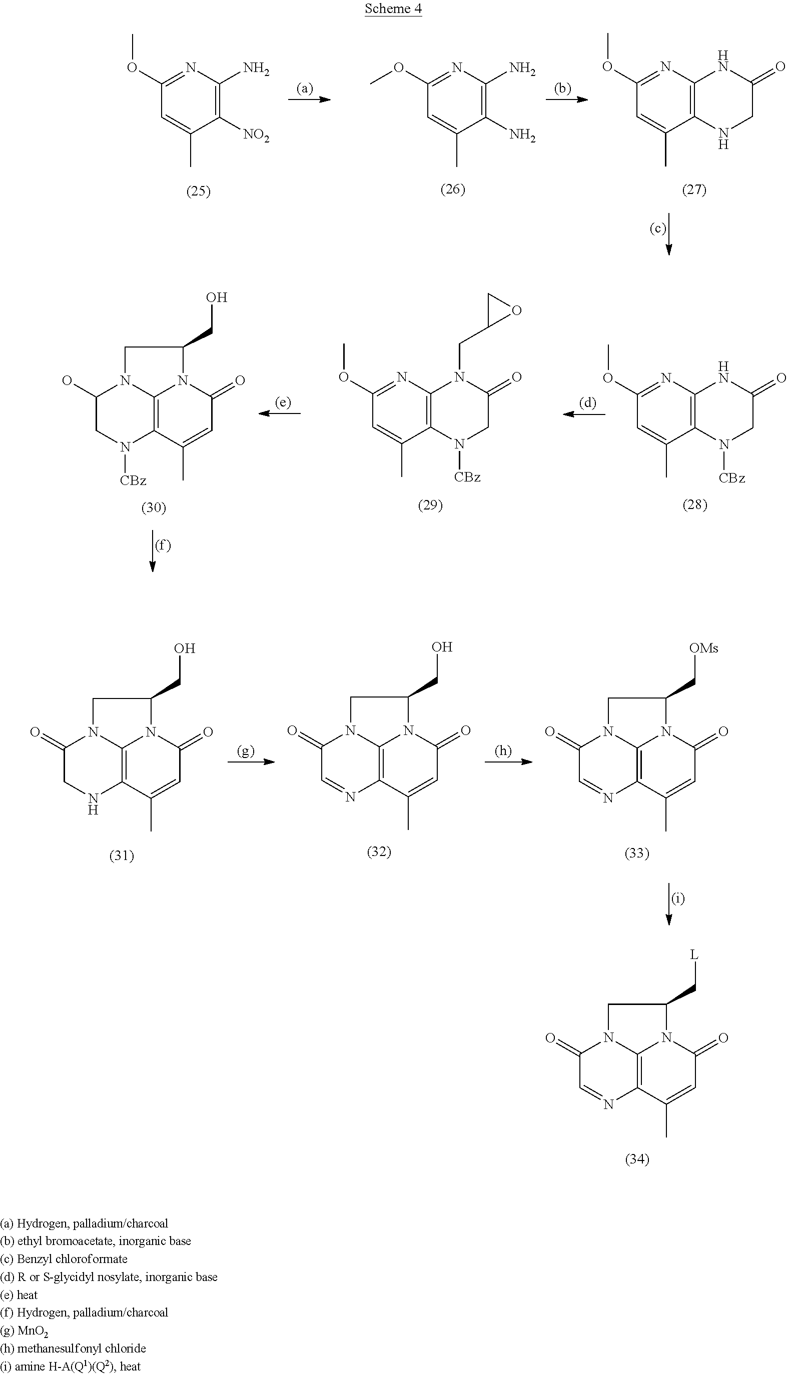

Reduction of aminonitropyridine (24) under standard hydrogenation conditions leads to bis-aniline (25). Alkyation with ethyl bromoacetate followed by cyclisation with potassium tert-butoxide gives (26). This is protected with a carboxybenzyl group to give (27) which can then be reacted with (commercially available) S-glycidyl nosylate ((2S)-2-oxiranylmethyl 3-nitrobenzenesulfonate) to give (28). Cyclisation under thermal conditions gives (29). Hydrogenolysis of the CBz group (30) and subsequent oxidation with manganese(II)oxide (31), followed by mesylation and displacement with an appropriate amine gives representatives of (IIID) (34) (L is -A(Q¹)(Q²)).

Interconversions of R^{1a}, R^{1b}, R², A and R⁵ are conventional. In compounds which contain an optionally protected hydroxy group, suitable conventional hydroxy protecting groups which may be removed without disrupting the remainder of the molecule include acyl and alkylsilyl groups. N-protecting groups are removed by conventional methods.

Interconversion of R^{1a} and R^{1b} groups may be carried out conventionally, on compounds of formula (I). For example R^{1a} or R^{1b} methoxy is convertible to R^{1a} or R^{1b} hydroxy by treatment with lithium and diphenylphosphine (general method described in Ireland et al, J. Amer. Chem. Soc., 1973, 7829) or HBr. Alkylation of the hydroxy group with a suitable alkyl derivative bearing a leaving group such as halide, yields R^{1a} or R^{1b} substituted alkoxy. R^{1a} or R^{1b} halo such as bromo may be converted to cyano by treatment with copper (I) cyanide in N,N-dimethylformamide. R^{1a} or R^{1b} carboxy may be obtained by conventional hydrolysis of R^{1a} or R^{1b} cyano, and the carboxy converted to hydroxymethyl by conventional reduction.

Compounds of formula HA-N(R²⁰)R^{2′} are known compounds or may be prepared analogously to known compounds, see for example WO2004/035569, WO2004/089947, WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2003082835, WO2002026723, WO06002047 and WO06014580, WO06134378, WO06137485, WO07016610, WO07081597, WO07071936, WO07115947, WO07118130, WO07122258, WO08006648, WO08003690 and WO08009700, each incorporated by reference herein in their entirety.

Further details for the preparation of compounds of formula (I) are found in the examples.

The antibacterial compounds according to the invention may be formulated for administration in any convenient way for use in human or veterinary medicine, by analogy with other antibacterials compounds.

The pharmaceutical compositions of the invention may be formulated for administration by any route and include those in a form adapted for oral, topical or parenteral use and may be used for the treatment of bacterial infection in mammals including humans.

The compositions may be in the form of tablets, capsules, powders, granules, lozenges, suppositories, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration, and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats; emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid; and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material (e.g. compound of formula (I) or pharmaceutically acceptable salt and/or N-oxide thereof), depending on the method of administration. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. In some embodiments from about 1.5 to about 50 mg active/kg patient body weight is administered per day. Suitably the dosage is from 5 to 30 mg/kg per day.

The compound of formula (I) may be the sole therapeutic agent in the compositions of the invention or a combination with other antibacterials including antitubercular compounds. If the other antibacterial is a β-lactam then a β-lactamase inhibitor may also be employed.

Compounds of formula (I) may be used to inhibit the growth of one or more of a wide range of organisms including both Gram-negative and Gram-positive organisms, including, for example, one or more of:

Hemophilus influenzae,
Klebsiella pneumoniae,
Legionella pneumoniae
Pseudomonas aeruginosa,
Escherichia coli,
Proteus mirabilis,
Enterobacter cloacae,
Enterobacter aerogenes,
Serratia marcescens,
Helicobacter pylori,
Salmonella enteritidis,
Salmonella typhi,
Acinetobacter baumanii,
Neisseria gonorrhoeae,
Neisseria meningitides,
Moraxella catarrhalis,
Mycobacterium tuberculosis,
Streptococcus pneumoniae,
Streptococcus pyogenes,
Staphylococcus aureus,
Enterococcus faecalis,
Enterococcus faecium,
Chlamydia pneumoniae, and
Staphylococcus epidermidis.

Some compounds of formula (I) may be active against more than one organism.

In some embodiments, compounds of the invention have activity against one or more of:

Hemophilus influenzae,
Klebsiella pneumoniae,
Pseudomonas aeruginosa,
Escherichia coli,
Proteus mirabilis,
Enterobacter cloacae,
Enterobacter aerogenes,
Moraxella catarrhalis,
Mycobacterium tuberculosis,
Streptococcus pneumoniae,
Streptococcus pyogenes,
Staphylococcus aureus,
Enterococcus faecalis, and
Enterococcus faecium.

Compounds of formula (I) may therefore be used in the treatment of bacterial infections caused by a wide range of organisms including both Gram-negative and Gram-positive organisms, including the above-listed organisms, such as upper and/or lower respiratory tract infections, skin and soft tissue infections and/or urinary tract infections, including for example tuberculosis caused by Mycobacterium tuberculosis.

Antibacterial activity may be determined by the methods described herein.

The following examples illustrate the preparation of certain compounds of formula (I) and the activity of certain compounds of formula (I) against various bacterial organisms.

### EXAMPLES AND EXPERIMENTAL

### General

### Abbreviations in the Examples:

MS=mass spectrum
ES=Electrospray mass spectroscopy
LCMS/LC-MS=Liquid chromatography mass spectroscopy
HPLC=high performance liquid chromatography
rt=room temperature
Rf=retention factor

Certain reagents are also abbreviated herein. TFA refers to trifluoroacetic acid, THF refers to tetrahydrofuran, Pd/C refers to palladium on carbon catalyst, DCM refers to dichloromethane, MeOH refers to methanol, DMF refers to dimethylformamide, EtOAc refers to ethylacetate, DDQ refers to 2,3-dichloro-5,6-dicyano-1,4-benzoquinone, NaBH(OAc)₃ refers to sodium triacetoxyborohydride, Pd₂(dba)₃ refers to tris(dibenzylideneacetone)dipalladium (0).

Proton nuclear magnetic resonance (¹H NMR) spectra were recorded at 400 or 250 MHz, and chemical shifts are reported in parts per million (ppm) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, td=triplet of doublets, app=apparent, br=broad. J indicates the NMR coupling constant measured in Hertz. CDCl₃ is deuteriochloroform and CD₃OD is tetradeuteriomethanol. Mass spectra were obtained using electrospray (ES) ionization techniques. All temperatures are reported in degrees Celsius.

MP-carbonate refers to macroporous triethylammonium methylpolystyrene carbonate (Argonaut Technologies). Amberlyst® A21 is a weakly basic, macroreticular resin with alkyl amine functionality, ®Registered trademark of Rohm & Haas Co.

AD mix alpha is prepared by mixing potassium osmate (K₂OsO₄.2H₂O) (0.52 g), (3a,9R,3′″a,4′″b,9′″R)-9,9′-[1,4-phthalazinediylbis(oxy)]bis[6′-(methyloxy)-10,11-dihydrocinchonan] [(DHQ)₂PHAL] (5.52 g), then adding potassium ferricyanide [K₃Fe(CN)₆] (700 g) and powdered potassium carbonate (294 g). This mixture is stirred in a blender for 30 minutes. This provides approximately 1 kg of AD mix alpha, which is commercially available from Aldrich. See K. Barry Sharpless et al, J. Org. Chem., 1992, 57 (10), 2771. AD mix beta is the corresponding mixture prepared with (9S,9′″S)-9,9′-[1,4-phthalazinediylbis(oxy)]bis[6′-(methyloxy)-10,11-dihydrocinchonan] [(DHQD)₂PHAL]. Where AD mix alpha/beta is referred to, this is a 1:1 mixture of the alpha and beta mix.

Celite® is a filter aid composed of acid-washed diatomaceous silica, and is a trademark of Manville Corp., Denver, Colo.

SCX Cartridge is an ion exchange column containing strong cation exchange resin (benzene sulfonic acid) supplied by Varian, USA.

Chiralpak IA and Chiralpak AS-H are polysaccharide based chiral HPLC columns (Chiral Technologies Inc.). Chiralpak AS-H column comprise amylose tris [(S)-alpha-methylbenzylcarbamate) coated onto 5 μm silica. Chiralpak IA column comprise silica for preparative column (5 μm particle size, 21 mm ID×250 mm L) immobilized with Amylose tris (3,5-dimethylphenylcarbamate). Chiralpak AD and AD-H columns comprise silica for preparative columns (5 μm particle size AD-H and 10 μm particle size AD, 21 mm ID×250 mm L; 20 μM particle size AD, 101 mm ID×250 mm L) coated with Amylose tris (3,5-dimethylphenylcarbamate) (Chiral Technologies USA). Measured retention times are dependent on the precise conditions of the chromatographic procedures. Where quoted below in the Examples they are indicative of the order of elution. Kromasil 5 micron C-18 column (21 mm×250 mm) comprises octadecylsilane chemically bonded to 5 micron porous silica gel.

As will be understood by the skilled chemist, references to preparations carried out in a similar manner to, or by the general method of, other preparations, may encompass variations in routine parameters such as time, temperature, workup conditions, minor changes in reagent amounts etc.

Reactions involving metal hydrides including lithium hydride, lithium aluminium hydride, di-isobutylaluminium hydride, sodium hydride, sodium borohydride and sodium triacetoxyborohydride are carried out under argon or other inert gas.

### Example 1

### (1R)-1-({4-[(3,4-dihydro-2H-pyrano[2,3-c]pyridin-6-ylmethyl)amino]-1-piperidinyl}methyl)-6-methyl-1,2-dihydro-4H,9H-imidazo[1,2,3-4]-1,8-naphthyridine-4,9-dione hydrochloride

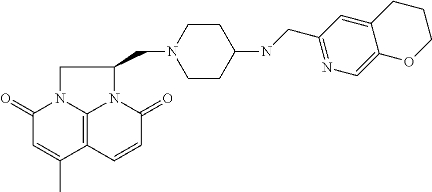

### (a) N-[3-bromo-6-(methyloxy)-2-pyridinyl]-2,2-dimethylpropanamide

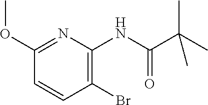

A solution of 2,2-dimethyl-N-[6-(methyloxy)-2-pyridinyl]propanamide (10 g, 48.0 mmol) in tetrahydrofuran (100 ml) under Argon was cooled to −78° C. and then treated with n-butyllithium (42.3 ml, 106 mmol, 2.5M solution in hexanes) over 20 minutes. The reaction was then allowed to warm to 0° C. and was stirred at 0° C. for 4 h. The clear reaction went yellow and then a solid crushed out. The mixture was then recooled to −78° C. and treated with 1,2-dibromoethane (4.97 ml, 57.6 mmol) over 5 minutes. The solution was allowed to warm to room temperature and was stirred at room temperature for 30 minutes. Water (20 mL) was then carefully added followed by further water (80 mL) and the mixture was extracted with diethyl ether (3×100 mL).

Combined organics were dried (MgSO₄), filtered and evaporated to give the crude product which was dissolved in warm ethyl acetate (20 mL) and allowed to stand in the freezer overnight. The solid that crystallised out was filtered off, washed with ice-cooled diethyl ether (˜5 mL) and dried in vacuo to afford 6.9 g of the desired product as a white solid (50%).

The filtrate was evaporated to afford ˜5 g of crude which was purified by silica chromatography (0-15% EtOAc/40-60 petr. ether) to afford more of the desired product (2.45 g, 17.8%).

MS (ES+) m/z 287/289 (MH⁺)

### (b) Ethyl (2E)-3-[2-[(2,2-dimethylpropanoyl)amino]-6-(methyloxy)-3-pyridinyl]-2-butenoate N2479-61-A2 and ethyl 3-[2-[(2,2-dimethylpropanoyl)amino]-6-(methyloxy)-3-pyridinyl]-3-butenoate

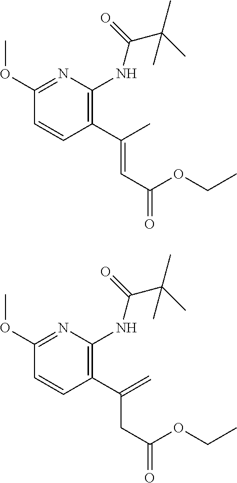

A mixture of N-[3-bromo-6-(methyloxy)-2-pyridinyl]-2,2-dimethylpropanamide (8.25 g, 28.7 mmol), bis(tri-t-butylphosphine)palladium(0) (0.147 g, 0.287 mmol) and tris(dibenzylideneacetone)dipalladium(0) (0.526 g, 0.575 mmol) in dry, degassed 1,4-Dioxane (150 ml) was treated with ethyl (2E)-2-butenoate (4.29 ml, 34.5 mmol) and dicyclohexyl(methyl)amine (6.70 ml, 31.6 mmol) at room temperature under Argon. The reaction mixture was heated at reflux (the colour of the reaction changed from burgundy to yellow). After 2 h there was still 12% of product so the reaction was stirred at reflux overnight. There was still some starting material (9%) present but reaction was getting messy so the solvent was removed and water (250 mL) was added and extracted with diethyl ether (3×250 mL). Combined organics were dried (MgSO₄), filtered and evaporated to afford 9.4 g of crude which was purified by silica chromatography (0-35% EtOAc-40-60 pet.ether) to afford ethyl (2E)-3-[2-[(2,2-dimethylpropanoyl)amino]-6-(methyloxy)-3-pyridinyl]-2-butenoate as an orange oil (1.85 g, 20.1%). MS (ES+) m/z 321(MH⁺)

Also ethyl 3-[2-[(2,2-dimethylpropanoyl)amino]-6-(methyloxy)-3-pyridinyl]-3-butenoate was obtained as a yellow oil (0.8 g, 8.7%)

MS (ES+) m/z 321(MH⁺)

### (c) Ethyl 3-[2-[(2,2-dimethylpropanoyl)amino]-6-(methyloxy)-3-pyridinyl]butanoate

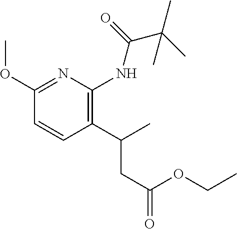

Ethyl 3-[2-[(2,2-dimethylpropanoyl)amino]-6-(methyloxy)-3-pyridinyl]-3-butenoate (0.8 g, 2.497 mmol) was dissolved in ethanol (50 ml) at room temperature and then treated with palladium on carbon (0.9 g, 10% paste). Everything was stirred at room temperature under 1 atm of hydrogen over the weekend. The reaction was complete so it was filtered through a celite pad and the pad was washed with more ethanol (150 mL). The ethanol was evaporated to afford the desired product as a white solid (0.75 g, 93%). MS (ES+) m/z 323(MH⁺)

### (d) Ethyl 3-[2-[(2,2-dimethylpropanoyl)amino]-6-(methyloxy)-3-pyridinyl]butanoate

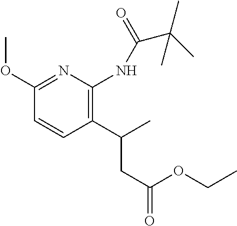

Ethyl (2E)-3-[2-[(2,2-dimethylpropanoyl)amino]-6-(methyloxy)-3-pyridinyl]-2-butenoate (1.85 g, 5.77 mmol) was dissolved in ethanol (100 ml) at room temperature and then treated with palladium on carbon (1.5 g, 10% paste). Everything was stirred at room temperature under 1 atm of hydrogen over the weekend. The reaction was complete so it was filtered through a celite pad and the pad was washed with more ethanol (100 mL). The ethanol was evaporated to afford the product as a white solid (1.72 g, 92%).

MS (ES+) m/z 323 (MH⁺)

### (e) Ethyl 3-[2-[(2,2-dimethylpropanoyl)amino]-6-(methyloxy)-3-pyridinyl]butanoate

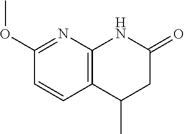

A mixture of ethyl 3-[2-[(2,2-dimethylpropanoyl)amino]-6-(methyloxy)-3-pyridinyl]butanoate (2.82 g, 8.75 mmol) in hydrochloric acid (5M) (30 ml, 150 mmol) was heated at 80° C. There was no starting material left after 3 h so the reaction was cooled, treated with water (30 mL), transferred to a conical flask and neutralised with potassium carbonate. The aqueous was then extracted with 20% methanol/DCM (3×80 mL). Combined organics were dried (MgSO₄), filtered and evaporated to afford the desired product as a white solid (1.36 g, 81%).

MS (ES+) m/z 193 (MH⁺)

### (f) 4-Methyl-7-(methyloxy)-1-[(2R)-2-oxiranylmethyl]-3,4-dihydro-1,8-naphthyridin-2(1H)-one

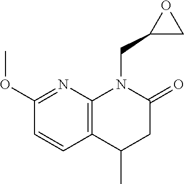

4-methyl-7-(methyloxy)-3,4-dihydro-1,8-naphthyridin-2(1H)-one (1.36 g, 7.08 mmol) was dissolved in N,N-Dimethylformamide (30 ml) at room temperature under nitrogen to give a yellow solution. The solution was then cooled with an ice bath and treated with sodium hydride (0.340 g, 8.49 mmol, 60% in mineral oil). The solution turned orange and after 10 minutes the ice bath was removed. After 20 minutes (2S)-2-oxiranylmethyl 3-nitrobenzenesulfonate (1.926 g, 7.43 mmol) was added. After 1 h all the starting material was consumed so the reaction was treated with a saturated aqueous solution of sodium bicarbonate (100 mL) and the aqueous was extracted with DCM (3×100 mL). The combined organic layers were dried (NaSO₄), filtered and evaporated to afford the desired product as a brown thick oil (1.9 g, 97%, 90% purity).

MS (ES+) m/z 249 (MH⁺)

### (g) (1S)-1-(hydroxymethyl)-6-methyl-1,2,5,6-tetrahydro-4H,9H-imidazo[1,2,3-ij]-1,8-naphthyridine-4,9-dione

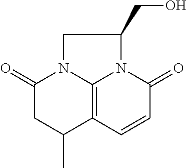

4-Methyl-7-(methyloxy)-1-[(2R)-2-oxiranylmethyl]-3,4-dihydro-1,8-naphthyridin-2(1H)-one (1.9 g, 6.89 mmol) was dissolved in N,N-Dimethylformamide (15 ml) at room temperature and heated at 130° C. for 10.5 h.

The DMF was then evaporated and the residue dried under high vacuum to afford a brown gum which was treated with ethyl acetate (25 mL) and sonicated for 10 minutes. The solid that crushed out was filtered off, washed with 5 mL of ethyl acetate and dried under vacuum overnight to afford the desired product as a brown solid (1.39 g, 67%).

MS (ES+) m/z 235 (MH⁺)

The filter was washed with some methanol and DCM; the solvents were evaporated to afford 0.1 g of desired product as a brown solid (5%)

MS (ES+) m/z 235(MH⁺)

### (h) [(2S)-7-methyl-4,9-dioxo-1,2,8,9-tetrahydro-4H,7H-imidazo[1,2,3-ij]-1,8-naphthyridin-2-yl]methyl methanesulfonate

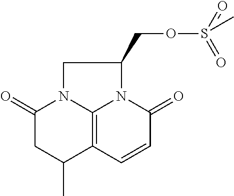

(1S)-1-(hydroxymethyl)-6-methyl-1,2,5,6-tetrahydro-4H,9H-imidazo[1,2,3-ij]-1,8-naphthyridine-4,9-dione (1.39 g, 4.75 mmol) was suspended in dichloromethane (100 ml) at room temperature under nitrogen and then treated with triethylamine (0.794 ml, 5.70 mmol). The mixture was then cooled using an ice-water bath. Methanesulfonyl chloride (0.444 ml, 5.70 mmol) was then added and the reaction allowed to warm up to room temperature. After 1 h there was still starting material present so 0.53 mL of triethylamine and 0.3 mL of methanesulfonyl chloride were added. After 3 h there was still starting material so 0.2 mL of triethylamine and 0.2 mL of methanesulfonyl chloride were added. After 1 h the reaction was complete so the mixture was washed with a saturated solution of sodium bicarbonate (100 mL); the aqueous was extracted with DCM (2×100 mL) and the combined organics were dried (NaSO₄), filtered and evaporated to afford the desired product as a brown gum (1.45 g, 98%).

Used without further purification.

MS (ES+) m/z 313(MH⁺)

### (i) 1,1-Dimethylethyl (1-{[(2R)-7-methyl-4,9-dioxo-1,2,8,9-tetrahydro-4H,7H-imidazo[1,2,3-ij]-1,8-naphthyridin-2-yl]methyl}-4-piperidinyl)carbamate

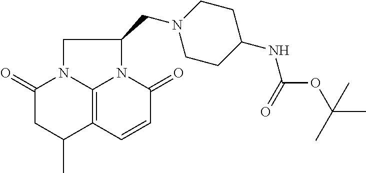

[(2S)-7-methyl-4,9-dioxo-1,2,8,9-tetrahydro-4H,7H-imidazo[1,2,3-ij]-1,8-naphthyridin-2-yl]methyl methanesulfonate (1.579 g, 5.06 mmol) was dissolved in dry Acetonitrile (100 ml) at room temperature under nitrogen and treated with pyridine (0.818 ml, 10.11 mmol). 1,1-dimethylethyl 4-piperidinylcarbamate (2.109 g, 10.11 mmol) was then added and the reaction was heated at 90° C. overnight. After night there was still 15% of starting material left so 0.5 g of 1,1-dimethylethyl 4-piperidinylcarbamate were added and the reaction was heated for 6 h. The solvent was then evaporated and the residue was partitioned between sat NaHCO₃ and DCM (100/100 mL). The layers were separated and the aqueous was extracted with DCM again (2×100 mL). Combined organics were dried (MgSO₄), filtered and evaporated to afford ˜3 g of crude which was purified by silica chromatography (0-10% MeOH/DCM) to afford the desired product as a yellow gum (666 mg, 31.6%).

MS (ES+) m/z 417 (MH⁺)

### (j) 1,1-Dimethylethyl (1-{[(1R)-6-methyl-4,9-dioxo-1,2-dihydro-4H,9H-imidazo[1,2,3-ij]-1,8-naphthyridin-1-yl]methyl}-4-piperidinyl)carbamate

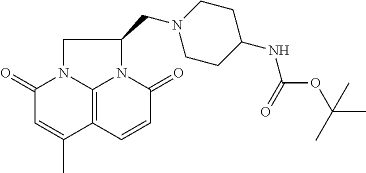

A solution of 1,1-dimethylethyl (1-{[(2R)-7-methyl-4,9-dioxo-1,2,8,9-tetrahydro-4H,7H-imidazo[1,2,3-ij]-1,8-naphthyridin-2-yl]methyl}-4-piperidinyl)carbamate (666 mg, 1.599 mmol) in dry degassed 1,4-Dioxane (50 ml) at rt under nitrogen was treated with DDQ (1089 mg, 4.80 mmol) and then heated at 80° C. for 2 h. LC-MS showed that the reaction was complete so the reaction was cooled to rt. The reaction mixture was treated with aqueous K₂CO₃ (5%, 100 mL) and with DCM (100 ml); the organic layer was separated and the aqueous was extracted with DCM (2×100 ml). The combined organic layers were then dried (NaSO₄), filtered and evaporated to give 550 mg of crude product as an orange solid. The crude was purified on a silica column (0-10% MeOH/DCM) to give 1,1-dimethylethyl (1-{[(1R)-6-methyl-4,9-dioxo-1,2-dihydro-4H,9H-imidazo[1,2,3-ij]-1,8-naphthyridin-1-yl]methyl}-4-piperidinyl)carbamate (450 mg, 0.977 mmol, 61.1% yield) as an orange solid.

MS (ES+) m/z 415 (MH⁺)

### (k) (1R)-1-[(4-amino-1-piperidinyl)methyl]-6-methyl-1,2-dihydro-4H,9H-imidazo[1,2,3-ij]-1,8-naphthyridine-4,9-dione dihydrochloride

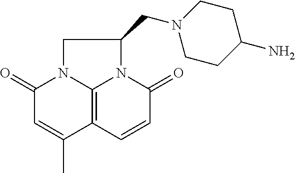

A solution of 1,1-dimethylethyl (1-{[(1R)-6-methyl-4,9-dioxo-1,2-dihydro-4H,9H-imidazo[1,2,3-ij]-1,8-naphthyridin-1-yl]methyl}-4-piperidinyl)carbamate (450 mg, 1.086 mmol) in chloroform (8 ml) at room temperature was treated with 4M HCl in dioxane (8 mL). A solid crushed out and the mixture was stirred at room temperature. After 0.5 h there was no starting material left so some methanol was added to dissolve most of the solid, followed by toluene and all the solvents were removed to afford (1R)-1-[(4-amino-1-piperidinyl)methyl]-6-methyl-1,2-dihydro-4H,9H-imidazo[1,2,3-ij]-1,8-naphthyridine-4,9-dione dihydrochloride (423 mg, 0.928 mmol, 86% yield) as a pale brown solid.

MS (ES+) m/z 314 (MH⁺)

### (I) Title Compound

A suspension of (1R)-1-[(4-amino-1-piperidinyl)methyl]-6-methyl-1,2-dihydro-4H,9H-imidazo[1,2,3-ij]-1,8-naphthyridine-4,9-dione dihydrochloride (50 mg, 0.135 mmol) in chloroform (4 ml) and methanol (0.200 ml) at room temperature under nitrogen was treated with triethylamine (0.057 ml, 0.406 mmol) and stirred for 0.25 h (the suspension turned into a solution). 3,4-Dihydro-2H-pyrano[2,3-c]pyridine-6-carbaldehyde (for a synthesis see WO2004058144, example 5(c)) (22.06 mg, 0.135 mmol) was then added and the reaction was stirred at room temperature for 0.5 h.

Sodium triacetoxyborohydride (90 mg, 0.406 mmol) was then added and the reaction was stirred at room temperature. After 2 h there was still some starting material so 30 mg of sodium triacetoxyborohydride were added. After 1 h sat NaHCO₃ (25 mL) was added followed by 20% MeOH/DCM (25 mL) and the aqueous was extracted and then separated from the organic layer. The aqueous was extracted again twice with 20% MeOH/DCM (2×25 mL). Combined organics were dried NaSO₄, filtered and evaporated to afford the crude. The crude was purified by silica chromatography (0-20% MeOH/DCM) to afford 41 mg of desired compound (65.7%) as a pale yellow gum. ¹H NMR δH CDCl₃, (400 MHz) 1.30-1.45 (m, 2H), 1.80-1.89 (m, 2H), 1.99-2.05 (m, 2H), 2.15-2.4 (m, 5H), 2.45-2.58 (m, 1H), 2.62-2.67 (m, 2H), 2.74-2.78 (m, 2H), 2.96 (d, 1H), 3.05-3.09 (m, 1H), 3.79 (s, 2H), 4.19-4.22 (m, 2H), 4.28-4.33 (m, 1H), 4.51-4.55 (m, 1H), 4.95-5.05 (m, 1H), 6.15 (s, 1H), 6.26 (d, 1H), 6.97 (s, 1H), 7.56 (d, 1H), 8.07 (s, 1H).

MS (ES+) m/z 462 (MH⁺).

The compound was dissolved in a small amount of MeOH/DCM and treated with 1 eq of a 1M solution of HCl in diethyl ether. The solvents were removed and the solid was dried in the desiccator (in the presence of P₂O₅) overnight to afford the mono-HCl salt of the product as a yellow solid (45.9 mg, 64.8%). LCMS was consistent with product.

### Example 2

### (1R)-1-({4-[(2,3-dihydro[1,4]oxathiino[2,3-c]pyridin-7-ylmethyl)amino]-1-piperidinyl}methyl)-6-methyl-1,2-dihydro-4H,9H-imidazo[1,2,3-ij]-1,8-naphthyridine-4,9-dione hydrochloride

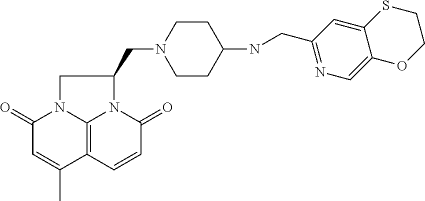

A suspension of (1R)-1-[(4-amino-1-piperidinyl)methyl]-6-methyl-1,2-dihydro-4H,9H-imidazo[1,2,3-ij]-1,8-naphthyridine-4,9-dione (50 mg, 0.135 mmol) (2HCl salt) in Chloroform (4 ml) and Methanol (0.200 ml) at room temperature under nitrogen was treated with triethylamine (0.057 ml, 0.406 mmol) and stirred for 0.25 h (the suspension turned into a solution). 2,3-dihydro[1,4]oxathiino[2,3-c]pyridine-7-carbaldehyde (for a synthesis see WO2004058144, Example 60) (24.50 mg, 0.135 mmol) was then added and the reaction was stirred at room temperature for 0.5 h. Sodium triacetoxyborohydride (90 mg, 0.406 mmol) was then added and the reaction was stirred at room temperature. After 2 h there was still some starting material so 30 mg of sodium triacetoxyborohydride were added. After 1 h sat NaHCO₃ (25 mL) was added followed by 20% MeOH/DCM (25 mL) and the aqueous was extracted and then separated from the organic layer. The aqueous was extracted again twice with 20% MeOH/DCM (2×25 mL). Combined organics were dried (NaSO₄), filtered and evaporated to afford the crude. The crude was purified by silica chromatography (0-20% MeOH/DCM) to afford 44 mg of desired compound (67.9%) as a pale yellow solid.

¹H NMR δH CDCl₃, (400 MHz) 1.25-1.45 (m, 2H), 1.79-1.88 (m, 2H), 2.19-2.40 (m, 5H), 2.43-2.54 (m, 1H), 2.62-2.67 (m, 2H), 2.96 (d, 1H), 3.05-3.09 (m, 1H), 3.15-3.18 (m, 2H), 3.76 (s, 2H), 4.28-4.33 (m, 1H), 4.39-4.42 (m, 2H), 4.51-4.55 (m, 1H), 4.95-5.05 (m, 1H), 6.14 (s, 1H), 6.26 (s, 1H), 6.99 (s, 1H), 7.55 (d, 1H), 8.01 (s, 1H).

MS (ES+) m/z 480 (MH⁺).

The compound was dissolved in a small amount of MeOH/DCM and treated with 1 eq of a 1M solution of HCl in Et2O. The solvents were removed and the solid was dried in the desiccator (P₂O₅) overnight to afford the mono-HCl salt of the product as a yellow solid (48.5 mg, 66%). LCMS was consistent with product.

### Example 3

### (1R)-1-({4-[(2,3-dihydro[1,4]dioxino[2,3-c]pyridin-7-ylmethyl)amino]-1-piperidinyl}methyl)-6-methyl-1,2-dihydro-4H,9H-imidazo[1,2,3-ij]-1,8-naphthyridine-4,9-dione hydrochloride

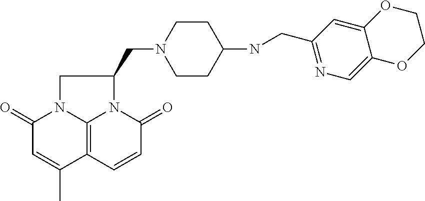

A suspension of (1R)-1-[(4-amino-1-piperidinyl)methyl]-6-methyl-1,2-dihydro-4H,9H-imidazo[1,2,3-ij]-1,8-naphthyridine-4,9-dione (50 mg, 0.135 mmol) (2HCl salt) in chloroform (4 ml) and methanol (0.200 ml) at room temperature under nitrogen was treated with triethylamine (0.057 ml, 0.406 mmol) and stirred for 0.25 h (the suspension turned into a solution). 2,3-dihydro[1,4]dioxino[2,3-c]pyridine-7-carbaldehyde (22.33 mg, 0.135 mmol) (for a synthesis see WO2004058144 Example 2(c) or WO03/087098 Example 19(d)) was then added and the reaction was stirred at room temperature for 0.5 h. Sodium triacetoxyborohydride (90 mg, 0.406 mmol) was then added and the reaction was stirred at room temperature. After 1.5 h sat NaHCO₃ (25 mL) was added followed by 20% MeOH/DCM (25 mL) and the aqueous was extracted and then separated from the organic layer. The aqueous layer was extracted again twice with 20% MeOH/DCM (2×25 mL). Combined organics were dried (NaSO₄), filtered and evaporated to afford the crude. The crude was purified by silica chromatography (0-20% MeOH/DCM) to afford 44.2 mg of desired compound (70.5%) as an off-white solid. ¹H NMR δH CDCl₃, (400 MHz) 1.28-1.45 (m, 2H), 1.80-1.89 (m, 2H), 2.19-2.34 (m, 5H), 2.45-2.55 (m, 1H), 2.62-2.67 (m, 2H), 2.96 (d, 1H), 3.05-3.09 (m, 1H), 3.78 (s, 2H), 4.27-4.34 (m, 5H), 4.51-4.585 (m, 1H), 4.95-5.05 (m, 1H), 6.15 (s, 1H), 6.26 (d, 1H), 6.81 (s, 1H), 7.56 (d, 1H), 8.10 (s, 1H).

MS (ES+) m/z 464 (MH⁺).

The compound was dissolved in a small amount of MeOH/DCM and treated with 1 eq of a 1M solution of HCl in Et₂O. The solvents were removed and the solid was dried in the desiccator (P₂O₅) overnight to afford the mono-HCl salt of the product as an off-white solid (46.9 mg, 65.9%). LCMS was consistent with product.

### Example 4

### (1R)-6-methyl-1-({4-[([1,3]oxathiolo[5,4-c]pyridin-6-ylmethyl)amino]-1-piperidinyl}methyl)-1,2-dihydro-4H,9H-imidazo[1,2,3-4]-1,8-naphthyridine-4,9-dione hydrochloride

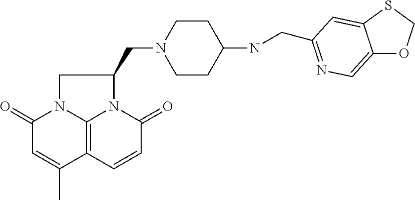

A suspension of (1R)-1-[(4-amino-1-piperidinyl)methyl]-6-methyl-1,2-dihydro-4H,9H-imidazo[1,2,3-ij]-1,8-naphthyridine-4,9-dione (50 mg, 0.135 mmol) (2HCl salt) in Chloroform (4 ml) and Methanol (0.200 ml) at room temperature under nitrogen was treated with triethylamine (0.057 ml, 0.406 mmol) and then stirred for 0.25 h (the suspension turned into a solution). [1,3]oxathiolo[5,4-c]pyridine-6-carbaldehyde (22.60 mg, 0.135 mmol) (for a synthesis see WO2004058144 Example 61) was then added and the reaction was stirred at room temperature for 0.5 h.

Sodium triacetoxyborohydride (90 mg, 0.406 mmol) was then added and the reaction was stirred at room temperature. After 1.5 h sat NaHCO₃ (25 mL) was added followed by 20% MeOH/DCM (25 mL) and the aqueous was extracted and then separated from the organic layer. the aqueous was extracted again twice with 20% MeOH/DCM (2×25 mL). Combined organics were dried (NaSO₄), filtered and evaporated to afford the crude. The crude was purified by silica chromatography (0-20% MeOH/DCM) to afford 44.2 mg of desired product (70.5%) as an off-white solid.

¹H NMR δH CDCl₃, (400 MHz) 1.20-1.45 (m, 2H), 1.70-2.00 (m, 2H), 2.15-2.40 (m, 5H), 2.45-2.55 (m, 1H), 2.60-2.70 (m, 2H), 2.95 (d, 1H), 3.05-3.15 (m, 1H), 3.80 (s, 2H), 4.25-4.35 (m, 1H), 4.50-4.60 (m, 1H), 4.95-5.05 (m, 1H), 5.62 (s, 2H), 6.15 (s, 1H), 6.26 (d, 1H), 7.20 (s, 1H), 7.56 (d, 1H), 8.00 (s, 1H).

MS (ES+) m/z 466 (MH⁺).

The compound was dissolved in a small amount of MeOH/DCM and treated with 1 eq of a 1M solution of HCl in Et2O. The solvents were removed and the solid was dried in the desiccator (P₂O₅) over the weekend to afford the mono-HCl salt of the product as an off-white solid (48.1 mg, 67.3%). LCMS was consistent with product.

### Biological Activity

### Antimicrobial Activity Assay:

Whole-cell antimicrobial activity was determined by broth microdilution using the Clinical and Laboratory Standards Institute (CLSI) recommended procedure, Document M7-A7, “Methods for Dilution Susceptibility Tests for Bacteria that Grow Aerobically”. The compounds were tested in serial two-fold dilutions ranging from 0.016 to 16 mcg/ml.

The minimum inhibitory concentration (MIC) was determined as the lowest concentration of compound that inhibited visible growth. A mirror reader was used to assist in determining the MIC endpoint.

Compounds were evaluated against Gram-positive organisms, including Staphylococcus aureus, Streptococcus pneumoniae, Streptococcus pyogenes, Enterococcus faecalis and Enterococcus faecium.

In addition, compounds were evaluated against Gram-negative organisms including Haemophilus influenzae, Moraxella catarrhalis, Escherichia coli, Pseudomonas aeruginosa, Proteus mirabilis, Enterobacter cloacae, Enterobacter aerogenes, Klebsiella pneumoniae and Stenotrophomonas maltophilia.

Each of the listed Examples, as identified in the present application was tested in the exemplified salt form. The tested Examples had a MIC≦2 μg/ml against a strain of at least one of the organisms listed above. For at least one strain of every organism listed above, at least one Example had a MIC≦2 μg/ml.

Mycobacterium tuberculosis H37Rv Inhibition Assay

The measurement of the minimum inhibitory concentration (MIC) for each tested compound was performed in 96 wells flat-bottom, polystyrene microtiter plates. Ten two-fold drug dilutions in neat DMSO starting at 400 μM were performed. Five μl of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μgml⁻¹ was prepared and 5 μl of this control curve was added to 95 μl of Middlebrook 7H9 (Difco catalogue Ref. 271310)+ADC medium (Becton Dickinson Catalogue Ref. 211887). (Row 11, lines A-H). Five μl of neat DMSO were added to row 12 (growth and Blank controls).

The inoculum was standardised to approximately 1×10⁷ cfu/ml and diluted 1 in 100 in Middlebrook 7H9+ADC medium and 0.025% Tween 80 (Sigma P4780), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value.

The listed Examples 1-4 were tested in the Mycobacterium tuberculosis H37Rv inhibition assay. Examples 1, 2 and 4 showed an MIC value of lower than 2.0 μg/ml. Example 3 showed an MIC value of >2.5 μg/ml.

What is claimed is:

1. A compound of formula (I): 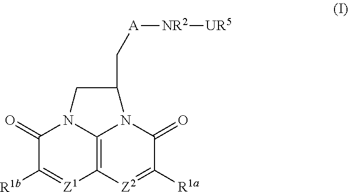
wherein:
one of Z¹ and Z² is CR^{1c} and the other is CH or N;
R^{1a} and R^{1b} are independently selected from hydrogen; halogen; cyano; (C₁₋₆)alkyl; (C₁₋₆)alkylthio; trifluoromethyl; trifluoromethoxy; carboxy; (C₁₋₆)alkoxy; hydroxy; hydroxy optionally substituted with (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; or aminocarbonyl;
wherein the amino group of the aminocarbonyl is optionally substituted by one or two (C₁₋₄)alkyl;
R^{1c} is (C₁₋₆)alkyl;
R² is hydrogen, (C₁₋₄)alkyl, or together with R⁶ forms Y as defined below;
A is a group (i) selected from: 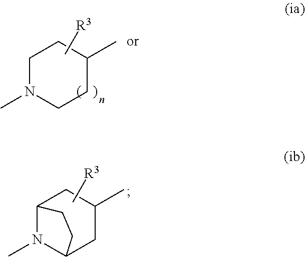
wherein:
R³ is independently as defined for R^{1a} and R^{1b} or is oxo; and
n is 1 or 2; or
A is a group (ii): 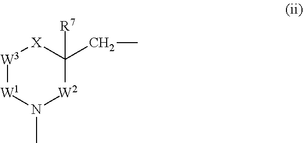
wherein:
W¹, W² and W³ are CR⁴R⁸
or W² and W³ are CR⁴R⁸ and W¹ represents a bond between W³ and N;
X is O, CR⁴R⁸, or NR⁶;
one R⁴ is independently as defined for R^{1a} and R^{1b} and the remainder and R⁸ are hydrogen or one R⁴ and R⁸ are together oxo and the remainder are hydrogen;
R⁶ is hydrogen or (C₁₋₆)alkyl; or together with R² forms Y;
R⁷ is hydrogen; halogen; (C₁₋₆)alkoxy; hydroxy; or (C₁₋₆)alkyl;
Y is CR⁴R⁸CH₂; CH₂CR⁴R⁸; (C═O); CR⁴R⁸; CR⁴R⁸(C═O); or (C═O)CR⁴R⁸; where R⁴ and R⁸ are independently as defined above;
or when X is CR⁴R⁸, R⁸ and R⁷ together represent a bond;
U is selected from CO and CH₂;
R⁵ is an optionally substituted bicyclic carbocyclic or heterocyclic ring system (B) containing up to four heteroatoms in each ring: 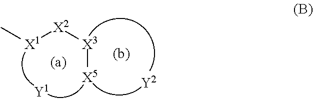
wherein:
at least one of rings (a) and (b) is aromatic;
X¹ is C or N when part of an aromatic ring, or CR¹⁴ when part of a non-aromatic ring;
X² is N, NR¹³, O, S(O)_{X}, CO or CR¹⁴ when part of an aromatic or non-aromatic ring or is CR¹⁴R¹⁵ when part of a non aromatic ring;
X³ and X⁵ are independently N or C;
Y¹ is a 0 to 4 atom linker group, each atom of which is independently selected from N, NR¹³, O, S(O)_{X}, CO or CR¹⁴ when part of an aromatic or non-aromatic ring or is CR¹⁴R¹⁵ when part of a non aromatic ring;
Y² is a 2 to 6 atom linker group, each atom of Y² being independently selected from N, NR¹³, O, S(O)_{X}, CO, CR¹⁴ when part of an aromatic or non-aromatic ring or is CR¹⁴R¹⁵ when part of a non aromatic ring;
wherein:
each x as defined above independently is 0, 1 or 2;
each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or R¹⁴ and R¹⁵ is oxo;
each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl.
wherein the amino group of the aminocarbonyl optionally mono or disubstituted by (C₁₋₄)alkyl;
or
a pharmaceutically acceptable salt and/or N-oxide thereof; and
provided that:
R^{1a} and R^{1b} are H when Z² or Z¹ is N, respectively.

2. A compound according to claim 1, wherein:
Z¹ is CR^{1c} and Z² is CH;
Z¹ is CH and Z² is CR^{1c};
Z¹ is CR^{1c} and Z² is N; or
Z¹ is N and Z² is CR^{1c}.

3. A compound according to claim 1, wherein R^{1a} is hydrogen and R^{1b} is hydrogen.

4. A compound according to claim 1, wherein R^{1c} is methyl.

5. A compound according to claim 1, wherein A is (ia), n is 1 and R³ is H or hydroxy in the 3-position, A is (ii), X is CR⁴R⁸ and R⁸ is H and R⁴ is H or OH, or A is (ii), X is O, R⁷ is H and W¹, W² and W³ are each CH₂.

6. A compound according to claim 5, wherein A is piperidin-4-yl or pyrrolidin-4-ylmethyl.

7. A compound according to claim 1, wherein U is CH₂.

8. A compound according to claim 1, wherein R⁵ is an aromatic heterocyclic ring (B) having 8-11 ring atoms including 2-4 heteroatoms of which at least one is N or NR¹³ in which Y² contains 2-3 heteroatoms, one of which is S and 1-2 are N, with one N bonded to X³, or the heterocyclic ring (B) has ring (a) aromatic selected from optionally substituted benzo, pyrido, pyridazino and pyrimidino and ring (b) non aromatic and Y² has 3-5 atoms, including at least one heteroatom, with O, S, CH₂ or NR¹³ bonded to X⁵ where R¹³ is hydrogen or other than hydrogen, and either NHCO bonded via N to X³, or O, S, CH₂ or NH bonded to X³.

9. A compound according to claim 1, wherein R⁵ is selected from:
3-oxo-3,4-dihydro-2H-pyrido[3,2-b][1,4]oxazin-6-yl,
3-oxo-3,4-dihydro-2H-pyrido[3,2-b][1,4]thiazin-6-yl,
2,3-dihydro-[1,4]dioxino[2,3-c]pyridin-7-yl,
[1,3]oxathiolo[5,4-c]pyridin-6-yl,
6-fluoro-2,3-dihydro-1,4-benzodioxin-7-yl,
2,3-dihydro[1,4]oxathiino[2,3-c]pyridin-7-yl,
3,4-dihydro-2H-pyrano[2,3-c]pyridin-6-yl,
5-fluoro-2,3-dihydro-1,4-benzodioxin-7-yl,
5-carbonitro-2,3-dihydro-1,4-benzodioxin-7-yl and or
2,3-dihydro-benzo[1,4]dioxin-6-yl.

10. A compound according to claim 1, which is:
(1R)-1-({4-[(3,4-dihydro-2H-pyrano[2,3-c]pyridin-6-ylmethyl)amino]-1-piperidinyl}methyl)-6-methyl-1,2-dihydro-4H,9H-imidazo[1,2,3-ij]-1,8-naphthyridine-4,9-dione;
(1R)-1-({4-[(2,3-dihydro[1,4]oxathiino[2,3-c]pyridin-7-ylmethyl)amino]-1-piperidinyl}methyl)-6-methyl-1,2-dihydro-4H,9H-imidazo[1,2,3-ij]-1,8-naphthyridine-4,9-dione;
(1R)-1-({4-[(2,3-dihydro[1,4]dioxino[2,3-c]pyridin-7-ylmethyl)amino]-1-piperidinyl}methyl)-6-methyl-1,2-dihydro-4H,9H-imidazo[1,2,3-ij]-1,8-naphthyridine-4,9-dione;
(1R)-6-methyl-1-({4-[([1,3]oxathiolo[5,4-c]pyridin-6-ylmethyl)amino]-1-piperidinyl}methyl)-1,2-dihydro-4H,9H-imidazo[1,2,3-ij]-1,8-naphthyridine-4,9-dione;
or a pharmaceutically acceptable salt and/or N-oxide thereof.

11. A compound according to claim 10, which is:
(1R)-1-({4-[(3,4-dihydro-2H-pyrano[2,3-c]pyridin-6-ylmethyl)amino]-1-piperidinyl}methyl)-6-methyl-1,2-dihydro-4H,9H-imidazo[1,2,3-ij]-1,8-naphthyridine-4,9-dione hydrochloride;
(1R)-1-({4-[(2,3-dihydro[1,4]oxathiino[2,3-c]pyridin-7-ylmethyl)amino]-1-piperidinyl}methyl)-6-methyl-1,2-dihydro-4H,9H-imidazo[1,2,3-ij]-1,8-naphthyridine-4,9-dione hydrochloride;
(1R)-1-({4-[(2,3-dihydro[1,4]dioxino[2,3-c]pyridin-7-ylmethyl)amino]-1-piperidinyl}methyl)-6-methyl-1,2-dihydro-4H,9H-imidazo[1,2,3-ij]-1,8-naphthyridine-4,9-dione hydrochloride; and or
(1R)-6-methyl-1-({-4-[([1,3]oxathiolo[5,4-c]pyridin-6-ylmethyl)amino]-1-piperidinyl}methyl)-1,2-dihydro-4H,9H-imidazo[1,2,3-ij]-1,8-naphthyridine-4,9-dione hydrochloride.

12. A method for treating bacterial infections which comprises administering an effective amount of a compound according to claim 1 to a patient in need thereof.

13.-15. (canceled)

16. A pharmaceutical composition comprising a compound according to claim 1 and a pharmaceutically acceptable carrier.

17. A pharmaceutical composition comprising a compound according to claim 10 and a pharmaceutically acceptable carrier.
